(19) United States
(12) Patent Application Publication
(10) Pub. No.: US 2026/0090963 A1
(43) Pub. Date: Apr. 2, 2026

(54) Xanthommatin as Color Additive for Food, Beverage and Drug Compositions

(71) Applicant: Northeastern University, Boston, MA (US)

(72) Inventor: Leila Deravi, Cambridge, MA (US)

(21) Appl. No.: 19/414,228

(22) Filed: Dec. 9, 2025

Related U.S. Application Data
(63) Continuation-in-part of application No. 17/819,981, filed on Aug. 16, 2022, now Pat. No. 12,514,797.
Application No. 17/819,981 is a continuation of application No. 16/141,690, filed on Sep. 25, 2018, now Pat. No. 11,464,719.
(60) Provisional application No. 62/563,017, filed on Sep. 25, 2017.
(60) Provisional application No. 62/627,710, filed on Feb. 7, 2018.
(60) Provisional application No. 62/627,717, filed on Feb. 7, 2018.
(60) Provisional application No. 62/669,369, filed on May 9, 2018.

(51) Int. Cl. A61K 8/29; A61K 8/02; A61K 8/25; A61K 8/26; A61K 8/41; A61K 8/49; A61K 8/73; A61K 8/81; A61K 8/85; A61K 8/86; A61Q 17/04; A61Q 19/08; C09B 19/00; C09B 61/00; C09B 67/02

(52) U.S. Cl. CPC A61K 8/29; A61K 8/0245; A61K 8/0275; A61K 8/0283; A61K 8/25; A61K 8/26; A61K 8/411; A61K 8/49; A61K 8/73; A61K 8/8152; A61K 8/85; A61K 8/86; A61Q 17/04; A61Q 19/08; C09B 19/00; C09B 61/00; C09B 67/0097; A61K 2800/412; A61K 2800/438; A61K 2800/45; A61K 2800/52; A61K 2800/651; A61K 2800/654

(57) ABSTRACT

A substrate having an edible color additive within the substrate or coated on the substrate is disclosed. The edible color additive comprises xanthommatin at a concentration of about 0.01% wt to about 0.5% wt and optionally a dry based carrier or a solution-based carrier. The substrate is a food, beverage, drink, medication or a drug form. The color additives of the disclosure can be produced in the color spectrum of yellow, orange, red, or concentrated forms (e.g., concentrated yellow appears as brown).

### RELATED APPLICATIONS

This application continuation-in-part of U.S. application Ser. No. 17/819,981, filed Aug. 16, 2022, which is a continuation of U.S. application Ser. No. 16/141,690, filed Sep. 25, 2018, now U.S. Pat. No. 11,464,719, issued on Oct. 11, 2022, which claims the benefit of U.S. Provisional Application No. 62/563,017, filed on Sep. 25, 2017, 62/627,710, filed on Feb. 7, 2018, 62/627,717, filed on Feb. 7, 2018 and 62/669,369, filed on May 9, 2018. The entire teachings of the above applications are incorporated herein by reference.

### GOVERNMENT SUPPORT

This invention was made with government support under Grant No. W911NF-16-1-0455 by the Army Research Office. The government has certain rights in the invention.

### BACKGROUND

The standard inorganic oxide pigments used in coatings and in the cosmetic industries typically lack color richness (chroma) and variety of hues. One approach to address this limitation has been to use organic pigments which offer more diversity in color; however, some disadvantages of using organic pigments are their limited hiding power, weak color stability, poor dispersion ability and poor weather durability. To improve pigment stability, dispersion and weather durability, organic pigments can be encapsulated within mica and other inorganic material that provide color, luster, iridescence, color travel and texture to the designated formulation. Another approach is to make pearlescent or sparkling “effect” pigments which are typically platelets (5-50 μm diameters) comprising mica or mica coated with iron oxides; however, some disadvantages of these materials include uncontrollable variance in platelet thickness and dispersion, the presence of impurities and heterogeneity in size/shapes. Further, many cosmetics that feature effect pigments are limited to tinted cover-ups that only impart color, which can often cake onto skin and accentuate the presence of imperfections in the skin by highlighting blemishes and unevenly collecting in enlarged pores and fine lines. There is a need for compositions, including cosmetic and dermatological compositions and adjuvants, which provide improved hiding power, improved dispersion ability and improved weather durability, and that can impart luminosity and diffuse reflectance of light while still being stable when excited by all wavelengths of solar light (ultraviolet [UV] through short-wave infrared light). Yet, most solar radiation protectants, for example, in cosmetic compositions, leave select regions of visible (400-490 nm) and infrared (750-2500 nm) light unfiltered. These sources of carcinogenic solar energy should not be ignored. Excessive exposure to infrared radiation has been demonstrated to increase matrix metalloprotease 1 (MMP-1) activity that leads to the destruction of collagen fibers resulting in the formation of coarse wrinkles. UVB radiation (280-320 nm) accounts for 0.5% of all incident solar light and induces modifications to the genomic DNA of keratinocytes and melanocytes in the epidermal skin layer; while, UVA radiation (320-400 nm) accounts for 99.5% of solar light, and together with UVB, enhances the production of reactive oxygen species (ROS) within both epidermal and dermal layers. Visible light, specifically blue light (440-490 nm), has also been noted to lead to the over production of reactive oxygen species (ROS) that can be attributed to photo-induced aging. Therefore, there is also a need for compositions, including cosmetic and dermatological compositions and adjuvants, which provide improved solar protection, including protection from infrared radiation and visible light, and/or suppression of ROS formation.

The ability to switch color (or peak reflected wavelength) in response to different environmental stimuli is also of great interest for protective pigments and personal care applications, as it enables the ability to turn “on” an active color or color filter on demand. However, existing color switching materials, such as those based on utilizing inorganic compounds (e.g. prussian blue, vanadium oxide, nickel oxide, tungsten), small organic molecules (e.g. viologen), and conductive polymers (e.g. polyaniline, polyimide, poly(3,4-ethylenedioxythiophene), poly(3,4-ethylenedioxypyrrole)) are not suitable for topical, cosmetic applications, as they are often dispersed in an organic (toxic) medium prior to application. Therefore, there is also a need for safe compositions that provide color, and color tunability in response to pH and electrochemical (or chemical redox) triggers.

### SUMMARY

Compositions are provided which have a number of advantages, for example, when designed as cosmetic and dermatological compositions and adjuvants, they can provide improved hiding power, improved dispersion ability and improved weather durability, and can impart luminosity and diffuse reflectance of light while still being stable when excited by all wavelengths of solar light. Further, they can provide improved solar protection, including protection from infrared radiation and visible light, and/or suppression of ROS formation. Yet further, they can provide color, and color tunability in response to pH and electrochemical (or chemical redox) triggers.

One embodiment is a composition including a plurality of synthetic particles having a size in the micrometer or nanometer range, each synthetic particle including one or more aggregates of a pigment selected from phenoxazone, phenoxazine, and a derivate or precursor thereof, and a stabilizing material which has a refractive index larger than 1.45; the aggregates having a size larger than about 100 nm and the composition being biodegradable and biocompatible.

A further embodiment is a composition comprising aggregates of a pigment selected from phenoxazone, phenoxazine, and a derivate or precursor thereof stabilized in a polyelectrolyte solution.

Yet a further embodiment is a method for extracting phenoxazone and/or

phenoxazine, comprising refluxing tissue containing ommochrome in a solvent.

Yet a further embodiment is a method for synthesizing ommochromes comprising electrochemically oxidizing 3-hydroxykynurenine.

Yet a further embodiment is a composition comprising (i) a colorant having an aromatic group and/or a chemical group allowing interaction with a pigment selected from phenoxazone, phenoxazine, and a derivate or precursor thereof, and (ii) the pigment; the pigment stabilizing the colorant; and the composition being biodegradable and biocompatible.

Yet a further embodiment is a composition comprising a solid and transparent matrix and (i) a free pigment selected from phenoxazone, phenoxazine, and a derivate or precursor thereof, and/or (ii) one or more aggregates of a pigment selected from phenoxazone, phenoxazine, and a derivate or precursor thereof, the free pigment and/or the aggregates being homogenously distributed throughout the matrix; and the composition being biodegradable and biocompatible.

Yet a further embodiment is a sensor comprising a composition described herein, wherein the material is positioned to allow physical contact with a sample.

Yet a further embodiment is a color-changing composition, comprising a composition described herein, the color-changing composition changing its color in response to changes in pH, humidity, solar light, and/or presence of chemical or electrical oxidizing or reducing agents.

The disclosure is also directed to the use of xanthommatin as an edible color additive that can impart colors from yellow to orange to red to an edible substrate depending upon the wavelength and pH. The substrate can be a food, beverage/drink or a medication/dosage form. In an embodiment is disclosed an edible substrate having an edible color additive within the substrate or coated on the substrate, comprising an edible color additive being xanthommatin at a concentration of about 0.01% wt to about 0.5% wt, and optionally a dry-based carrier or a solution-based carrier.

In another embodiment is disclosed a method of coating an edible substrate where the method comprises applying an edible color additive to the edible substrate, comprising an edible color additive being xanthommatin at a concentration of about 0.01% wt to about 0.5% wt, and optionally a dry-based carrier or a solution-based carrier.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

The patent or application file contains at least one drawing executed in color. Copies of this patent or patent application publication with color drawings will be provided by the Office upon request and payment of the necessary fee.

FIG. 1 provides a bar graph of average measured diameters of pigment aerosols from SEM micrographs plotted against the selected diameters from dynamic material analyzer, in which error bars indicate standard error, where N=82-135; the inset includes representative SEM micrographs of pigment aerosols (scale bar=500 nm).

FIG. 2 provides top-down SEM images of granule films. G1-G3 granule films indicate the increased density of granules, even though they are still ˜1 layer thick (scale bars are 5 μm). For multi-layer films G4-G6, representative cross-sectional SEM images show variations in thickness (scale for G4 is 1 μm; G5 is 5 μm, and G6 is 5 μm).

FIG. 3 provides an SEM image of natural squid granules (left) and four SEM images of synthetic granules with pigment (xanthommatin, also referred to herein as “Xa”), specifically, inorganic materials (silicon dioxide and titanium dioxide) and biodegradable polymers (PLGA and PSMA) which were used to stabilize Xa; these hybrid materials mimic the morphology of natural granules found within squid chromatophores.

FIG. 4 provides two flow diagrams of pigment extraction protocols. The prior method (top) features eight steps and requires approximately 6.5 hours to complete. The method according to a present embodiment (bottom) consists of five steps and can be completed within 1 hour.

FIG. 5 provides a schematic of the believed mechanism of the electrosynthesis of Xa (top) and a graph (bottom) showing the corresponding measured charge and current response over time as the electrochemical synthesis proceed (F is Faraday constant, Nₐ is Avogadro number, C is charge, n_{Xa} numbers of moles of Xa reacted, nₑ numbers of moles of electron consumed).

FIG. 6 provides two graphs with results of the UV-Visible Spectroscopy which was used to monitor the progression of the electrosynthesis of Xa. As illustrated in the top graph, 3-hydroxykyneurine has a lambda max at 380 nm, and a distinct peak at 454 nm was observed after reacting 3-hydroxykyneurine for 120 mins—the decrease in absorbance intensity over time was used as an indicator of the transformation to Xa which has a characteristic lambda max centered at 450 nm. The bottom graph illustrates the increase of absorbance centered at 454 nm with reaction time.

FIG. 7 provides an absorbance over elution time graph with results from HPLC which was used to identify the components of the reaction mixture over time of the electrosynthesis. Peak identity was assigned by considering the UV-visible absorbance profile of each peak. The first peak with approximate retention time 1.5 mins was identified as 3-hydroxykynuerine. Peak 2 and 3 at 7.2 and 7.5 mins respectively was associated with Xa. Over 2 hours the intensity of Peak 1 decreased as Peak 2 and 3 increased indicating the transformation of that reactant to the products.

FIG. 8 provides an intensity over wavelength graph illustrating measured variations in absorptive behaviors of Xa associated with an increasing pH.

FIG. 9 provides two graphs of the experimental determination of pKa, where absorbance intensity (collected at 430 nm, top, and 360 nm, bottom) was followed as a function of varying pH. The two observed pKas are indicated by the dashed lines at 5.50 and 8.40.

FIG. 10 provides a graph illustrating the switching responses of Xa recorded as a function of current density change within a multi-layered electrochromic device (ECD).

FIG. 11 provides two graphs showing absorption curves for three separate ECDs that were assembled by varying the ratio between Xa and PEDOT:PSS and switched from oxidized (O1, O2, O3; see top graph) to reduced (R1, R2, R3; see bottom graph) states.

FIG. 12 provides absorbance curves (see top graph) and corresponding comparator values (see table) of Xa to known UV-filters, avo- and oxy-benzone. All values have been normalized to same concentration (0.10 mM). X-1 represents synthetic Xa; X-2 represents Xa extracted from squid at 0.10 mM. SPF was calculated using the Mansur equation and critical wavelength was determined as the wavelength at which 90% of the absorbance curve resides.

FIG. 13 provides a graph of the calculated optical cross-section of pigment particles as a function of particle diameter, where solid lines are scattering cross-sections and dashed lines are absorption cross-sections. The inset is an expanded view of particle diameters 5-200 nm, indicating no difference between absorption and scattering at diameters <200 nm.

FIG. 14 illustrates the hydroxyl radical antioxidant capacity (HORAC) activity assay activity of Xa. The HORAC assay was used to evaluate the application of Xa as a natural antioxidant. Xa consumes hydroxyl radicals produced by the activation of the fenton reagent preventing the degradation of the fluorescein probe. At the above concentrations, Xa demonstrated enhanced activity in comparison to commercially available standard gallic acid, a known phenolic antioxidant, showing its potential as an effective, photostable antioxidant.

FIG. 15A shows xanthommatin's wavelength as a function of pH change in solution.

FIG. 15B shows the color of xanthommatin in an oxidized form (yellow) and a reduced form (red).

FIG. 16 shows an example of chocolate with xanthommatin at a concentration of 1 mg/mL incorporated into melted chocolate.

FIG. 17A shows xanthommatin at a concentration of 1 mg/mL incorporated into melted butter.

FIG. 17B shows the color change that occurs by the addition of xanthommatin (Xa; 1 mg/mL) mixed into melted butter and after the butter solidifies, compared to control without the addition of Xa.

FIGS. 18A and 18B show the color change that occurs by the addition of xanthommatin (Xa; 1 mg/mL) mixed into (A) a dark soda and (B) a colorless soda, compared to control without the addition of Xa.

FIG. 19 shows an example embodiment of DORITOS™ Cool Ranch™ Chips, soaked and dried in red Xa (1 mg/mL) (top left view), spray coated with 70% v/v ethanol/water (top right view), control chip without Xa (bottom left view), spray coated with a solution of Xa (1 mg/mL) in 70% v/v ethanol/water (bottom right view). A graph showing wavelength (nm) to reflectance (%) is shown for the Cool Ranch™ control and the Cool Ranch™ chip spray coated with Xa in 70% v/v ethanol and water.

FIGS. 20A, 20B and 20C show solutions of Xa (1 mg/mL) as a food color dye. FIG. 20A shows representative colors of Xa in solution; [1] Xa+DI water, [2] Xa+DI water+3 M ascorbic acid, [3] Xa+70% EtOH, [4] Concentrated red food dye, [5] Red food dye+DI water. FIG. 20B is a graph showing the wavelength (nm) to absorbance (O.D.) for the five samples of FIG. 20A. FIG. 20C shows the color changes that occur when Xa is mixed with green color food dye.

### DETAILED DESCRIPTION

A description of example embodiments follows.

A first embodiment is a composition comprising a plurality of synthetic particles having a size in the micrometer or nanometer range, each synthetic particle including one or more aggregates of a pigment selected from phenoxazone, phenoxazine, and a derivate or precursor thereof, and a stabilizing material which has a refractive index larger than 1.45; the aggregates having a size larger than about 100 nm and the composition being biodegradable and/or biocompatible.

In an aspect of the first embodiment, the stabilizing material is positioned between or among the aggregates to inhibit or prevent clumping of the aggregates. In another aspect of the first embodiment or any aforementioned aspect thereof, each synthetic particle is polymer encapsulated. In another aspect of the first embodiment or any aforementioned aspect thereof, the composition further comprises a transparent and biocompatible polymer. In another aspect of the aforementioned aspect, the transparent and biocompatible polymer is poly vinyl alcohol, poly methyl methacrylate, polyethylene glycol, poly lactic-co-glycolic acid, poly lactide, poly(butylene succinate), silicone-based polymers, or a derivative thereof. In another aspect of the first embodiment or any aforementioned aspect thereof, the pigment is 3-hydroxykynurenine, xanthommatin, ommatin D, dihydroxy-xanthommatin, rhodommatin, or a derivative thereof. In another aspect of the first embodiment or any aforementioned aspect thereof, the composition further comprises a transparent stabilizer mixed with the plurality of synthetic particles and having a refractive index larger than 1.45. In another aspect of the first embodiment or any aforementioned aspect thereof, the stabilizing material is a metal oxide, polymer, or bare mineral. In another aspect of the aforementioned aspect, the metal oxide is one of, or a blend of one or more of, silicon dioxide, titanium dioxide, iron oxide, aluminum oxide, and zinc oxide. In another aspect of the aforementioned aspect, the polymer is a polyamide, polyurethane, polyester, polysaccharide, polyethylene glycol, or polymethacrylate. In another aspect of the first embodiment or any aforementioned aspect thereof, the stabilizing material is titanium dioxide. In another aspect of the first embodiment or any aforementioned aspect thereof, the synthetic particle comprises poly lactic-co-glycolic acid. In another aspect of the first embodiment or any aforementioned aspect thereof, the synthetic particles have size from about 10 to about 100 micrometers. In another aspect of the first embodiment or any aforementioned aspect thereof, the synthetic particle has a core-shell structure, and one or more of the aggregates form the core. In another aspect of the first embodiment or any aforementioned aspect thereof, the synthetic particles are stabilized in a polyelectrolyte solution. In another aspect of the aforementioned aspect, the polyelectrolyte is a polyacid. In another aspect of the aforementioned aspect, the polyelectrolyte is polyacrylic acid, poly methyl methacrylate, poly(sodium styrene sulfonate), or poly(allylamine) hydrochloride. In another aspect of the first embodiment or any aforementioned aspect thereof, the aggregates make up about 0.01 to about 0.9% wt of the composition. In another aspect of the first embodiment or any aforementioned aspect thereof, the composition is a broad-spectrum absorber. In another aspect of the first embodiment or any aforementioned aspect thereof, the pigment is an ommochrome. In another aspect of the first embodiment or any aforementioned aspect thereof, the stabilizing material is a second pigment, different from the pigment, selected from phenoxazone, phenoxazine, and a derivate or precursor thereof.

A second embodiment is a composition comprising aggregates of a pigment selected from phenoxazone, phenoxazine, and a derivate or precursor thereof stabilized in a polyelectrolyte solution. In an aspect of the second embodiment, the polyelectrolyte is a polyacid. In another aspect of the second embodiment, the polyelectrolyte is polyacrylic acid, poly methyl methacrylate, poly(sodium styrene sulfonate), or poly(allylamine) hydrochloride.

A third embodiment is a method extracting phenoxazone and/or phenoxazine, comprising refluxing tissue containing ommochrome pigments in a solvent. In an aspect of the third embodiment, the tissue is homogenized. In an aspect of the third embodiment or an aforementioned aspect thereof, the solvent is acidic methanol.

In another aspect of the third embodiment or any aforementioned aspect thereof, the method extracts xanthommatin, decarboxylated xanthommatin, and/or dihydroxanthommatin.

In another aspect of the third embodiment or any aforementioned aspect thereof, the tissue is squid dermal tissue.

In another aspect of the third embodiment or any aforementioned aspect thereof, the method is a bulk extraction.

In another aspect of the third embodiment or any aforementioned aspect thereof, the method further purifies the phenoxazone and/or phenoxazine.

The embodiments for extraction allow extraction which can be about 6 to 7 times faster than previously reported methods. They also can allow production of about 40 to 50 times more purified phenoxazone and/or phenoxazine.

Pigments can be isolated in native granular form and/or in molecular form.

A fourth embodiment is a method for synthesizing ommochromes comprising electrochemically oxidizing tryptophan, formyl-kynurenine, kynurenine, and/or 3-hydroxykynurenine.

Ommochromes that can be synthesized using the described methods (e.g., of the fourth embodiment) include, but are not limited to xanthommatin decarboxylated xanthommatin, and dihydroxanthommatin, and ommins.

A fifth embodiment is a composition comprising a (i) colorant having an aromatic group and/or a chemical group allowing interaction with a pigment selected from phenoxazone, phenoxazine, and a derivate or precursor thereof, and (ii) the pigment; the pigment stabilizing the colorant; and the composition being biodegradable and biocompatible. Suitable colorants include oil soluble dyes (including D&C Yellow #11 or Red #17), water soluble dyes (including FD&C Blue #1 and #2 or Red #4), toners (including D&C Red #6 Sodium salt), true pigments (including D&C Red #30 or #36), and lakes (including aluminum and/or zirconium lake).

A sixth embodiment is a composition comprising a solid and transparent matrix and (i) a free pigment selected from phenoxazone, phenoxazine, and a derivate or precursor thereof, and/or (ii) one or more aggregates of a pigment selected from phenoxazone, phenoxazine, and a derivate or precursor thereof, the free pigment and/or the aggregates being homogenously distributed throughout the matrix; and the composition being biodegradable and biocompatible.

A seventh embodiment is a sensor comprising a composition described herein (for example, of the first, fifth, or sixth embodiment or any aforementioned aspect thereof), wherein the material is positioned to allow physical contact with a sample. In an aspect of the seventh embodiment, a topical sensor response to changes molecular structure and subsequent color in response to variations in pH, humidity, solar light and/or presence of chemical or electrical oxidizing or reducing agents in the sample. In another aspect of the seventh embodiment or any aforementioned aspect thereof, the material is further positioned such that a color change in response to the sample can be observed by a user of the sensor.

An eighth embodiment is a color-changing composition, comprising the composition described herein (for example, of the first, fifth, or sixth embodiment or any aforementioned aspect thereof), the color-changing composition changing its color in response to changes in pH, humidity, solar light, and/or presence of chemical or electrical oxidizing or reducing agents.

Pigments described herein (e.g., selected from phenoxazone, phenoxazine, and a derivate or precursor thereof (e.g., ommochrome pigments)) can be encapsulated or stabilized within synthetic particles having a size in the micrometer or nanometer range. They can further be stabilized within and/or outside a porous synthetic particle, optionally, followed by polymer encapsulation to secure the pigment in place.

The synthetic particles described herein, can be used to prepare a transparent polymer coating utilizing any biocompatible cross-linking polymer (e.g. poly vinyl alcohol, poly methyl methacrylate, poly ethylene glycol), which are widely used in daily use cosmetic and water resistance personal care applications.

In certain embodiments, the cosmetic compositions described herein, minimize the impacts on photo-induced aging and disease (i.e., they can be used as anti-aging cosmetics).

The compositions described herein can be used as total solar radiation protectants that can be used alone or in combination with other commercially available antioxidants and UV filters.

A further embodiment is an electrochromic device comprising a composition as described herein (e.g., as described in the above embodiments). In aspects of this embodiment, the composition is positioned in a layer or film. In further aspects of this embodiment or aspect thereof, the composition comprises a polymer matrix (e.g., a polymer (PEDOT-PSS) matrix). In further aspects of this embodiment or aspects thereof, the composition is capable of switching color in response to different applied voltages (e.g., a double potential step from +1.5 V to −1.5 V vs ground can be applied to the device). In further aspects of this embodiment or aspects thereof, the devices and composition is adapted and/or configured to allow for a redox-dependent color switch. Typically, the time for switching from the oxidized to the reduced state, upon application of a suitable potential is in the second range.

The compositions can be spectrally tuned, for example, by varying the ratio between PEDOT:PSS and pigment. For example, different concentrations of xanthommatin (e.g., from 0.04 mg/ml to 0.16 mg/ml) can be used while the amount of polymer matrix (e.g., PEDOT:PSS) remains constant.

A further embodiment is a method of changing the color of a color changing composition (e.g., a color changing topical treatment), comprising applying a reducing agent (e.g., ascorbic acid) onto a composition described herein (e.g., in the embodiments described above) which has been coated directly on soft tissue (e.g., facial tissue of a person) or synthetic tissue (e.g. hydrogels). A suitable reducing agent includes, but are not limited to, ascorbic acid (vitamin C).

As used herein, a “total solar radiation protectant” refers to a protectant (e.g., a composition described herein) which covers the 280 to 2500 nm wavelength range at film thicknesses <2 μm.

As used herein, “synthetic particle” refers to a structured material (either nanometer or micrometer sized) that is made in the laboratory using a chemical synthesis.

As used herein, “aggregates of a pigment” refers to a combination of two or more phenoxazone or phenoxazine-based compounds that form a three-dimensional structure that is stabilized through electrostatic, covalent, and/or non-covalent interactions.

As used herein, a “stabilizing material” refers to any substance that inhibits or prevents the physical or chemical alteration of a second material and/or eliminates the breakdown of another compositional discrete substance.

As used herein, “biodegradable” refers to a substance that can be decomposed, degraded, or converted by living systems.

As used herein, “biocompatible” refers to a substance that does not elicit an undesirable effect (infection, inflammation) when placed in contact with the human body.

As used herein, “inhibit or preventing clumping of the aggregates” refers to maintaining particles or aggregates that remain as discrete units in a suspension or when deposited as films.

As used herein, “polymer encapsulated” refers to a nano- or micro-particle which has an exterior polymer containing shell that is used to stabilize or encapsulate a material within the shell.

As used herein, “transparent” refers to a substance that does not substantially absorb or reflect light in the visible spectral regions (400-750 nm).

As used herein, a “transparent stabilizer” refers to a material (polymer, metal, or metal oxide) that does not absorb or reflect light in the visible spectral regions (400-750 nm).

As used herein, “refluxing” refers to boiling a solution, such that the liquid portion is vaporized and returned to the stock.

As used herein, “homogenized” refers to creating a homogenous mixture out of an originally insoluble or immiscible material.

As used herein, “electrochemically oxidizing” refers to generating a potential (voltage) gradient within a 2- or 3-electrode system (includes, reference Ag/AgCl electrode, counter (Pt) electrode, and working (e.g., reticulated vitreous carbon) electrode to induce a loss of electrons into the analyte of interest. Chemical redox refers to adding a soluble compound (ascorbic acid-reducer- and/or sodium nitrate-oxidizer) to change color.

As used herein, “interaction” between an aromatic group and/or a chemical group of a colorant with a pigment selected from phenoxazone, phenoxazine, and a derivate or precursor thereof, refers to pi stacking (π-π stacking) which are attractive but noncovalent forces between adjacent aromatic rings that contain conjugated aromatic pi bonds.

As used herein, “change of” or “changing” color refers to a spectral shift of at least 20 nm in the visible through short-wave infrared color space.

The pigments described herein (and compositions containing them) can not only be used as solar radiation filters, but can also be employed as reactive oxygen species scavengers and used to assist in anti-ageing applications.

The synthetic particles and compositions described herein have numerous commercial applications including as multi-functional colorants in cosmetics and other coating industries, as anti-ageing serums, creams, or topical cosmetics, as multi-functional sun-care materials, as antioxidants, as anti-aging skin care products, as blue light filters, as cosmetics, as personal care products, and/or as sun care products.

The compositions described herein can be used in cosmetics and/or dermatological formulations. In certain embodiments the compositions contain pigments (e.g., selected from a phenoxazone, phenoxazine, and a derivative or precursor thereof) which are UVA and UVB absorbers and the compositions can be used as sun-protectant products, alone or in combination with other compositions.

Pigments that can be used in the present embodiments include, but are not limited to, pigments selected from phenoxazone, phenoxazine, and a derivate or precursor thereof, for example, 3-hydroxykynurenine, xanthommatin, decarboxylated xanthommatin, dihydro-xanthommatin, rhodommatin, ommatin D, and ommins (e.g., ommin A). For example, the pigment can be xanthommatin

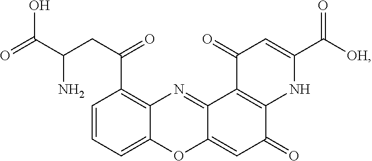 [SMILES: NC(CC(=O)c1cccc2oc3cc(=O)c4[nH]c(C(=O)O)cc(=O)c4c-3nc12)C(=O)O]

or a derivative thereof. For example, a derivative of xanthommatin can be decarboxylated xanthommatin

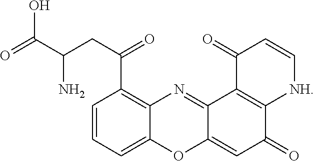 [SMILES: NC(CC(=O)c1cccc2oc3cc(=O)c4[nH]ccc(=O)c4c-3nc12)C(=O)O]

For example, the pigment can be ommin A

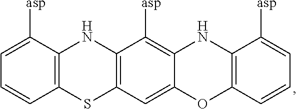

or a derivative thereof.

A pigment suitable for the embodiments described herein, can also be an ommochrome represented by structural formulas (I), (II), (III) or (IV)

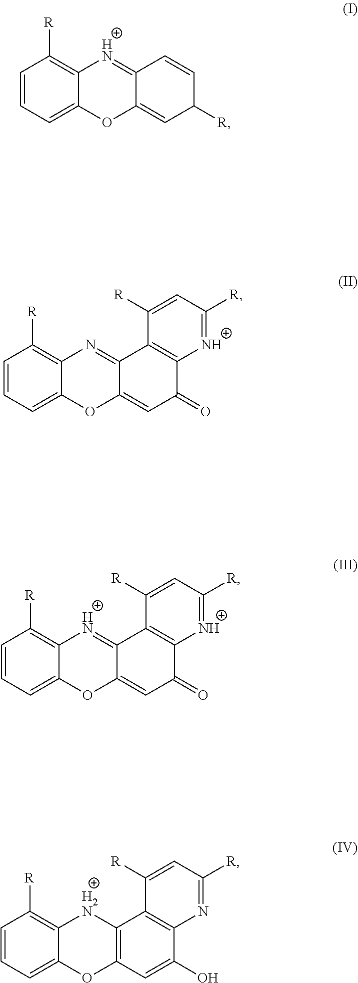

wherein R can be a proton donating or accepting group (including carboxylic acids or amines), a saturated or unsaturated functional group, another phenoxazine/phenoxazone moiety, or a combination of the above.

Both, xanthommatin and decarboxylated xanthommatin are highly conjugated organic molecules and have been identified in squid Doryteuthis pealeii chromatophore pigment granules. These pigments have a deep red color and contain combinations of xanthommatin and decarboxylated xanthommatin dyes. In solution, these pigments have an intrinsic UV absorbance and ability to scatter light—both are characteristics that contribute to brightening and/or distorting visible color. Specifically, these pigments are UVA and UVB absorbers, which makes them suitable for sun-protectant compositions. They can also be used as natural colorants.

Compositions described herein, when designed as cosmetic or dermatological formulation, typically comprise pigment at a concentration of 0.01-0.9% wt.

Suitable pigments can not only be extracted from cephalopods (e.g., squid Doryteuthis pealeii chromatophores), but also from other natural sources such as the eyes, integumentary system, organs, and eggs of arthropods. These pigments can also be synthesized using methods described herein or known in the art.

The compositions disclosed herein can be designed to have one or more aesthetic and functional properties (e.g., blurring, brightening, UV-absorber).

The compositions can be in a variety of forms, including but not limited to, a serum, cream, or loose powder.

When the compositions are in the form of a coating, they can be designed to impart complex directional differences and other well-controlled optical properties depending on thickness of coating.

In certain embodiments, the compositions provided herein, can contain colorants. The U.S. positive list (e.g. the list of color additives approved for use in cosmetics found in Title 21 of the Code of Federal Regulations Part 73, FDA) features a list of permitted colorants used in color cosmetics and includes synthetic certifiable organic colorants and non-synthetic organic and inorganic colorants. Natural colorants, which are pigments that are derived from animal, vegetable or mineral sources, can also additionally be included in the compositions described herein.

In certain embodiments, the compositions provided herein, can further contain pigments other than those selected from phenoxazone, phenoxazine, and a derivate or precursor thereof. For example, animal derived pigments such as carmine (also called cochineal) can be included. Cochineal extract has a bright red color obtained from the aluminum salt of carminic acid. The characteristic deep red color is produced from some insects such as the conchineal scale and certain Porphyrophora species. Carmine is the only organic colorant exempt from certification by the US FDA. Inorganic oxides, such as iron oxides, (yellow, red, brown) can also be included, for example, in the development of color cosmetics.

The compositions provided herein can be used in cosmetic formulations to enhance skin radiance and glow and provide angle dependent coloration. They can add dramatic visual effects by providing color, luster, iridescence, color travel (i.e. pigments can appear darker or brighter and/or change color at different viewing angles), and texture.

Known effect pigments constructed from mica, a type of phyllosillicate mineral that consists of psuedohexagonal crystals, or aluminum flakes can also be included. These minerals exhibit nearly perfect cleavage resulting in the formation of platelet particles of varying size. The typical platelet thickness ranges from 100-1000 nm and has the ability to show interference colors. In the production of pearlescent pigments, the optical layers are built upon the mica substrate by coating the surface with photoactive materials such as iron oxide or the new phenoxazine/phenoxazone-based pigments.

Other effect pigments can be constructed with transparent substrates such as borosilicate and silica. Each of these substrates provides a range of optical properties that can deliver differentiated appearance and performance characteristics in cosmetic formulations. Silica can be used as a substrate for pearls due to its tunable thickness, particle assembly and low refractive index (1.46) when compared to mica (1.58).

A significant difference between the refractive indices between the substrate and pigment is essential for a strong pearlescent effect. When films of different refractive indices are combined, multiple reflections result, and stronger interference colors can be achieved. This multilayer technique is demonstrated in nature and can be applied when designing new effect pigments.

Pigment particle size is important in designing effect pigments. The classical light scattering mechanisms such as Rayleigh scattering, Mie scattering and large particle Mie scattering are particle size dependent. Small particles are very efficient at scattering shorter light wavelengths; here nanoparticle scatter is highly dependent on wavelength with shorter wavelengths (ultraviolet or blue light) scattering more intensely than longer wavelengths (red or near-IR light). A key example of this phenomenon is highlighted with the incorporation of titanium dioxide nanoparticles in sunscreen products; titanium dioxide nanoparticles are frequently used in sun protectant products due to their ability to scatter hazardous UV radiation and thus protects the skin from the penetration of harmful radiation. Large particle Mie scattering occurs when the particle is larger than incident wavelength of light; in this case scattering is not wavelength dependent. In large particle Mie scattering a forward directed antenna lobe projecting in the general direction of the incident light results. Effect pigments can be 10-20 microns, which is much larger than the wavelengths of interest (UV and visible light), therefore, one can anticipate large particle Mie scattering.

The multifunctionality of modified D. pealeii pigments will be highlighted with its combinatorial UV absorbance and light scattering capability.

Further embodiments are as follows:

Phenoxazone-based biopigments and the derivatives thereof as antioxidants. Pigments include but are not limited to 3-hydroxykynurenine, xanthommatin (and its decarboxylated form) and ommatin D in a monomeric and polymeric forms.

The application of phenoxazone-based biopigments and their derivatives, including but not limited to 3-hydroxykynurenine, xanthommatin and ommatin D, as total solar protectants.

The application of phenoxazone-based biopigments and their derivatives, including but not limited to 3-hydroxykynurenine, xanthommatin and ommatin D, as the base ingredient for an anti-ageing topical treatment that targets free-radical scavenging.

The application of natural or biologically inspired nano- or micro-particles incorporating phenoxazone-based pigments and polymeric and/or protein complexes as total solar protectants.

Bio-hybrid nano- or micro-particles incorporating phenoxazone-based pigments and metal-oxides (mineral based, or metal-derived) as total solar protectants.

Bio-hybrid nano- or micro-particles incorporating phenoxazone-based pigments and metal-oxides (mineral based, or metal-derived) as anti-oxidants for anti-ageing applications.

Bio-hybrid nano- or micro-particles incorporating phenoxazone-based pigments and metal-oxides (mineral based, or metal-derived) as blue light filters.

The application of phenoxazone-based biopigments and their derivatives, including but not limited to 3-hydroxykynurenine, xanthommatin and ommatin D, as blue light filters.

The application of phenoxazone-based biopigments and their derivatives, including but not limited to 3-hydroxykynurenine, xanthommatin and ommatin D, as photostabilizers that enhance ultraviolet radiation (UVR) protection.

The application of phenoxazone-based biopigments and their derivatives, including but not limited to 3-hydroxykynurenine, xanthommatin and ommatin D, as filters that protect against UVA induced photoaging.

A synthetic strategy that incorporates phenoxazone-based biopigments and their derivatives, including but not limited to 3-hydroxykynurenine, xanthommatin and ommatin D, as bio-hybrid solar radiation filters.

A formulation of anti-aging cosmetic containing phenoxazone-based biopigments and their derivatives, including but not limited to 3-hydroxykynurenine, xanthommatin and ommatin D.

A formulation of anti-aging cosmetic containing phenoxazone-based biopigments and their derivatives, including but not limited to 3-hydroxykynurenine, xanthommatin and ommatin D, nanomaterials.

A formulation of anti-aging cosmetic containing phenoxazone-based biopigments and their derivatives, including but not limited to 3-hydroxykynurenine, xanthommatin and ommatin D, solar radiation filters.

A formulation of anti-aging cosmetic containing phenoxazone-based biopigments and their derivatives, including but not limited to 3-hydroxykynurenine, xanthommatin and ommatin D, and with biologically inspired nano- or micro-particles incorporating phenoxazone-based pigments and polymeric and/or protein complexes or metal-oxides (mineral based, or metal-derived) as solar radiation filters.

A cosmetic or dermatological formulation containing a redox active and/or UV-sensitive light protection filter materials chosen from the group of pigments consisting of ommochromes including xanthommatin, decarboxylated xanthommatin, dihydro-xanthommatin, rhodommatin, and ommatin D, and ommins and/or the natural chromatophore pigment granules isolated from cephalopod skin.

A method for stabilizing the formulation containing a redox active and/or UV-sensitive light protection filter materials chosen from the group of pigments consisting of ommochromes including xanthommatin, decarboxylated xanthommatin, dihydro-xanthommatin, rhodommatin, and ommatin D, and ommins isolated from cephalopod dermal tissue in native granular form and/or in molecular form as well as ommochromes synthesized via oxidation in vitro, comprising a step of adding a positively charged polyelectrolyte including but not limited to poly acrylic acid, gelatin, poly methyl methacrylate.

A pH sensor based on squid biochromes (both synthetic and naturally extracted forms) that is capable of color change between acidic (pH<3.00) and neutral (pH˜7) states.

An application for squid biochromes (both synthetic and naturally extracted forms) as a material for electrochromic devices where different applications of an applied electric field induce a “dynamic” color-changing palette for applications in coatings, paints, dyes, and apparel. Color change from yellow to red can be reversibly switched in the presence of oxidative and reducing potentials.

A green/color changing pigment that provides both visible and infrared coloration with color tunability while minimizing EHOS risk.

### Color Additives

Synthetic dyes of FD&C Red No. 40, FD&C Yellow No. 5, FD&C Yellow No. 6, FD&C Green No. 3, FD&C Blue No. 1, and FD&C Blue No. 2 are scheduled to be phased out in favor of naturally accessible color additives. Many natural alternatives, such as extracts from beets, carrots, paprika, and turmeric, may not be robust as a colorant in many applications.

The disclosure is directed to edible color additives comprising xanthommatin for imparting color or enhancing color of an edible substrate. Particularly preferred substrates are foods, drink/beverages and medication/drugs. The color additives of the disclosure can be imparted to the surface of the substrate, incorporated into/throughout/within the substrate, or as a coating on the substrate. The color additives of the disclosure comprising xanthommatin can be produced as extracts from natural sources of xanthommatin or the xanthommatin can be chemically synthesized. As shown in the examples herein, the color additives of the disclosure can be produced in the color spectrum of yellow, orange, red, or concentrated forms (e.g., concentrated yellow appears as brown).

As used herein, a “color additive” or “edible color additive” are used interchangeably herein and are defined as any dye, pigment of other substance which when added or applied to a food, drink, beverage or medicament is capable (alone or through reactions with other substances) of imparting color. A color additive is added to a substrate for a number of reasons, including but not limited to, creating a substrate in which the color additive mitigates or prevents the loss of or discoloration of the substrate due to expose to light, air, temperature extremes, moisture and storage conditions. The color additive can enhance the natural color of a substrate, change the color of a substrate, correct the natural variations in color of a substrate to produce a product of consistent appearance and quality. In embodiments, mixtures of color additives can be formed by mixing one color additive with one or more color additives or non-colored diluents without a chemical reaction. The color additive of the disclosure can be incorporated with other color additives of the disclosure to create new colors or the color can be enhanced with known colors and dyes.

In an embodiment, disclosed is an edible substrate having an edible color additive within the substrate or coated on the substrate comprising xanthommatin as the edible color additive at a concentration of about 0.01% wt to about 0.5% wt, and optionally a dry-based carrier or a solution-based carrier.

Xanthommatin can change color based on its protonation or oxidation state. The properties of xanthommatin apply directly to the edible color additive of this disclosure. The protonation state is dependent upon pH of a solution when xanthommatin is dissolved. When the solution is evaporated, the xanthommatin will retain the color of the solution in its solid form (e.g., if the solution is red, then the solidified xanthommatin is red). At a pH range from about pH 1.90 to about pH 3.50, the color of xanthommatin is an observable yellow. At a pH range from about pH 3.50 to about pH 8.30, the color of xanthommatin is an observable red. The color of xanthommatin can be modified by adjusting the properties for intended use.

Similarly, the oxidation state of xanthommatin can be determined based on if xanthommatin is positively charged, negatively charged, or in a neutral state, and will retain this state when the solution is removed (e.g., if the solution is red, then the solidified xanthommatin is red). When xanthommatin is oxidized (e.g., positively charged), xanthommatin is an observable yellow. When xanthommatin is reduced (e.g., negatively charged or neutral), xanthommatin is an observable red. The color of xanthommatin can be modified by adjusting the properties for intended use.

The color of the edible color additive can be further modified by adjusting the concentration of xanthommatin. The edible color additive is an observable brown at an increased concentration of xanthommatin when at lower concentrations it is an observable yellow. The color intensity is further changeable based on xanthommatin concentration in solution. The color of xanthommatin can be modified by adjusting the properties for intended use.

In some embodiments, the edible color additive absorbs light in a wavelength range of about 420 nm to about 480 nm. In some further embodiments, the edible color additive appears as yellow. In some embodiments, the edible color additive appears as brown.

In some embodiments, the edible color additive absorbs light in a wavelength range of about 620 nm to about 750 nm. In some further embodiments, the edible color additive appears as red.

In some embodiments, the edible color additive absorbs light in a wavelength range of about 590 nm to about 620 nm. In some further embodiments, the edible color additive appears as orange.

When the edible color additive is applied, coated, or mixed within a substrate that has a color, the edible color additive can boost, or enrich the color of a substrate or the substrate can boost or enrich the color of the edible color additive. The color boost or enrichment makes the color of the substrate, or the color additive appear more vivid to an observer. The edible color additive of this disclosure can also boost the color of other color additives. In a non-limiting example, when the color additive is mixed within a green solution, the color of the green solution increases the green intensity of the solution.

In some embodiments, the addition of the color additive to a substrate can darken the color of the substrate. In some embodiments, the addition of the color additive to a substrate result in a red shift in a reflectance spectrum.

Xanthommatin is a compound that also has electrostatic properties. These electrostatic properties assist with applying xanthommatin (and therefore the edible color additive) to a substrate. As used herein “electrostatic properties” is defined as how charges are distributed over a surface of volume of material that can exhibit polarization in a molecule (e.g., dipoles). The electrostatic properties allow for the edible colorant to adhere to the substrate, when the substrate is a solid.

As used herein, a “substrate” or “edible substrate” is defined herein as a food, beverage, drink, medication or a drug form. When the edible color additive is applied to a solid substrate, the edible color additive is also a solid and can be on the surface of the solid substrate or can be mixed into the solid substrate. When the edible color additive is applied to a liquid substrate (e.g., beverage, drink), the edible color additive is also a liquid and is dispersed uniformly throughout the liquid substrate.

As used herein, a “dry-based carrier” is defined herein as an edible solid that can contribute to texture, adhesion, flavor, smell, nutrients, antimicrobials, stability, improvement in handling, or a combination thereof when applied to a substrate. The dry-based carrier can be a powder or powder mix. The dry-based carrier can be applied to a substrate either separately or in tandem with the edible color additive. When applied in tandem with the edible color additive, the dry-based carrier can encapsulate and/or transport the edible color additive to the substrate. The dry-based carrier can be soluble in a liquid substrate and can be a solid on a solid substrate. The dry-based carrier can be coated onto a substrate with or without the edible color additive. The dry-based carrier can have electrostatic properties. The dry-based carrier can be independent of the edible color additive of the present disclosure.

Non-limiting examples of “dry-based carriers” can be a cheese powder, spices, monosodium glutamate (MSG), maltodextrin, salt, sugar, anti-caking agents, other edible color additives, or any combination thereof. In some embodiments, the anti-caking agents can be silicon dioxide (SiO₂), calcium silicate (CaSiO₃), magnesium carbonate (MgCO₃), magnesium silicate (MgSiO₃), sodium aluminosilicate (Na₂Al₂Si₁₄O₃₂·3H₂O), talc (Mg₃Si₄O₁₀(OH)₂), tricalcium phosphate (TCP, Ca₃(PO₄)₂), or combination thereof. The type of dry ingredients that can be used will depend on the edible substrate to be coated and is determined by the formulation or recipe.

As used herein, a “solution-based carrier” is defined herein as an edible liquid that dissolves or disperses the edible color additive onto/into a substrate for aiding in uniform application of the edible color additive. The solution-based carrier can be applied to a substrate separately or in tandem with the edible color additive. When applied in tandem with the edible color additive, the solution-based carrier can dissolve or suspend the edible color additive and transport the edible color additive to the substrate. The solution-based carrier can evaporate after transporting the edible color additive when applied to a solid substrate, which can form a film of the edible color additive on the surface of the substrate. The evaporation of the solution-based carrier can result in the loss of the solution-based carrier, or gelling (e.g., increase in viscosity of the solution-based carrier, becomes an amorphous state) of the solution-based carrier. The solution-based carrier can aid in dispersing the edible color additive in a liquid substrate. When applied to a liquid substrate, the solution-based carrier is a liquid. The solution-based carrier can have electrostatic properties. The solution-based carrier can be independent of the edible color additive of the present disclosure.

Non-limiting examples of “solution-based carriers” can be water, glycerin, propylene glycol, oils, alcohols, or any combination thereof. In some embodiments, oils can be, but are not limited to, olive oil, canola oil, coconut oil, vegetable oil, vegetable oil blends, peanut oil, sunflower oil, grapeseed oil, avocado oil, walnut oil, or any combination thereof. In some embodiments, alcohols can be, but are not limited to, methanol, ethanol, or a combination thereof. The type of solution that can be use will depend on the edible substrate and is determined by the formulation or recipe.

In one embodiment, the color additives can be added, applied, or coated to any food in which it is intended to enhance the color or appearance of the food, such as, but not limited to, confections (e.g., icing, frosting, sprinkles), candy, baked goods (e.g., cakes, cupcakes, cookies, pastries), cereal, fruit (e.g., canned fruits, preserved fruits, dried fruits, oranges, sauces like apple and cranberry, frozen fruits), meats and fish (e.g., sausages, hot dogs, salmon), condiments (e.g., ketchup, mustard, relish, pickles, hot sauces, salsa), spreads, salad dressings, snack foods (e.g., chips, pretzels, extruded snacks, popcorn, crackers), vegetables (e.g., canned vegetables, dried vegetables, fresh vegetables and produce, frozen vegetables), dairy (e.g., yogurts, cheeses, whipped creams), grains (e.g., pasta, rice), seasoning and flavorings, or sauces.

In another embodiment, the color additives can be added into a drink or beverage to enhance the color or appearance of the drink or beverage, such as, but not limited to, sports drinks, energy drinks, flavored milk, soda, juices, beverage mixes, electrolyte drinks, drink powders, protein shakes, concentrates, alcoholic beverages, liqueurs, nutritional shakes and drinks, carbonated beverages, acidic beverages, or water.

In yet another embodiment, the color additives can be added, applied, or coated to any medication or drug in which it is intended to enhance the color or appearance of the medication or drug, such as, but not limited to, cough syrups and other syrups, tablets, caplets, gel dosage forms, gummy dosage forms, or vitamins.

In some instances, the edible color additive including xanthommatin may use extracted xanthommatin or synthesized xanthommatin. In some further instances, synthesized xanthommatin may be labelled as vegetarian and/or vegan friendly. In some other instances, extracted xanthommatin cannot be labelled as vegetarian and/or vegan friendly. In some instances, the edible color additive is vegetarian and/or vegan friendly.

The color additives of the disclosure can be combined with edible coatings to enhance the flavor and/or appearance of a substrate or provide an edible barrier to protect or preserve the substrate from environmental deterioration of the substrate under conditions such as light, temperature variations, gas and/or moisture barrier, or to aid in adhesion of the edible coating to the substrate. In addition to the color additives of the disclosure, the edible barrier can comprise mono-, di- and polysaccharides such as starches and maltodextrin, proteins, lipids, flavoring agents, and other ingredients such as flavoring agents, spices, seeds. The ingredients comprising the edible coating will be understood by the skilled person based upon the specifications of the final product. Applying the edible color additive as a coating can be beneficial for the food industry, particularly coating solid, edible substrates. Coating an edible substrate is a process that applies a liquid, powder, or film to an edible substrate that can have benefits of enhancing the taste, texture, and/or appearance of the edible substrate. A coating can be applied to an edible substrate through methods that include, but are not limited to, dipping, immersing, spraying, dusting, sprinkling, pan coating, drum coating, extruding, coextruding or a combination thereof. These coatings can be further stabilized by using methods of drying, cooling, freezing, crystallizing, or thermal treatments (e.g., heating). Coatings can have additional benefits that include sensory improvement, moisture and shelf-life control, nutrient or additive delivery, mechanical protection, and/or processing advantages.

This disclosure is also directed to a method of coating an edible substrate, the method comprising applying xanthommatin as an edible color additive at a concentration of about 0.01% wt to about 0.5% wt to the edible substrate, and optionally a dry-based carrier or a solution-based carrier.

The color additive is colorfast or does not change color when applied as a coating to a substrate. Without being bound by theory, the coating of the color additive is stable due to the presence of xanthommatin in the color additive being a solid when coated onto a substrate. In a solid or dry form, the color additive does not have its oxidation state or protonation state affected and does not change color, making this storage stable. In some embodiments, the edible color additive is stable for up to two years. In some embodiments, the color additive stabilizes the color of the substrate as a coating. In some embodiments, the coating prevents degradation of the substrate (e.g., colorfast, storage stability).

Due to the electrostatic properties of xanthommatin, the color additives of the disclosure are particularly well-suited for coating applications for applying a film (e.g., continuous film coat) or coating to substrates such as foods, medicines and drug forms. Known coating technologies can be utilized for imparting a color coating onto the surface of a substrate and preferred coating methodologies will be selected depending upon the substrate. Coating techniques include but are not limited to electrostatic coating, natural coating formulations for fruits, vegetable and other foods, spray coating applications, immersion or dipping (partially or fully dipping or immersing the substrate and then drying the coated substrate), drum coating (e.g., tumble coating a plurality of items), pan coating, fluidized bed coating (for uniform distribution of particulates), enrobing, dusting, edible coatings like breading, hand-coating, extrusion. Such processes can be batch or continuous processes and will depend on production scale. Further, the carrier selected to deliver the color additive of the disclosure can vary depending upon the substrate, as will be discussed below.

In one embodiment, an electrostatic coating process can be used to coat a substrate such as a chip, a snack food, crackers, corn or other grain chips to finish the product in its commercial form. Electrostatic coating uses positive and negative charges to attract a coating (usually fine powders like spices, salt, or powdered sugar) to the substrate. In an non-limiting example, a corn chip can be made and coated with the color additive (for visual appeal) in a powdered form with optional other product finishing ingredients such as but limited to seasoning blends comprising one or more of flavor compounds (cheese powder, spices, MSG, etc.), maltodextrin (helps the powder stick and carries flavors), spices, salt, sugar, anti-caking agents to maintain powder flow. Other coating methods may be used in combination with an electrostatic coating process to augment or improve the adherence of the color additive and the product finishing ingredients to the substrate.

In another embodiment, the color additive of the disclosure can be added to natural coatings that are commonly used to preserve the shelf life of fruits, vegetables, meats, cheeses, baked goods. Such natural coatings include, lipids, waxes (carnauba, beeswax), proteins like soy and whey, pectin, starches, essential oils, and combinations of these, to provide an environmental and/or antimicrobial barrier to the coated substrate.

In yet another embodiment, the color additive of the disclosure and other optional ingredients can be incorporated into a solution-based carrier for spray coating onto a substrate, dipping the substrate or immersing the substrate into the solution-based carrier. A powdered form of the color additive of the disclosure is dissolved in a solution-based carrier/solvent to form a solution. Suitable solution-based carriers/solvents include, but are not limited to, water, glycerin, propylene glycol, oils, alcohols (e.g., ethanol, methanol), acidic methanol, an acidic solution, or any combination thereof. The concentration of ethanol will depend on the substrate and the conditions for finishing the product. For example, the solution-based carrier/solvent is about 50% v/v ethanol to about 50% v/v water to about 80% v/v ethanol to about 20% v/v water. In some instances, the solution-based carrier/solvent is about 50% v/v ethanol to about 50% v/v water. In some instances, the solution-based carrier/solvent is about 55% v/v ethanol to about 45% v/v water. In some instances, the solution-based carrier/solvent is about 60% v/v ethanol to about 40% v/v water. In some instances, the solution-based carrier/solvent is about 65% v/v ethanol to about 35% v/v water. In some instances, the solution-based carrier/solvent is about 70% v/v ethanol to about 30% v/v water. In some instances, the solution-based carrier/solvent is about 75% v/v ethanol to about 25% v/v water. In some instances, the solution-based carrier/solvent is about 80% v/v ethanol to about 20% v/v water.

As used herein, the term “about” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Example section herein.

In yet another embodiment, the color additive of the disclosure can be used in a fluidized air bed coating process. This process is useful for coating small substrates by suspending the substrate (food items) in a stream of moving air while the coating solution is sprayed onto them. The process allows for the precise application of the color additive resulting in a uniform coating or shell around the substrate, while simultaneous drying the coating.

For medications and drug dosage forms, the color additives of the disclosure can be applied using known techniques in the industry depending on the dosage form, active pharmaceutical ingredient (API) and other inactive ingredients, and intended use of the dosage form. Such coating techniques include, but are not limited to, sugar coating, fluidized air bed coating (e.g., Wurster coating), dry coating or compression coating, film coating produced by spray coating, electrostatic coating. The color additives of the disclosure can be formulated with other ingredients for the coating, such as, but not limited to, lipids and waxed (e.g., carnauba wax), anti-adherents/Glidants (prevent sticking such as talc), polymers (e.g., hydroxypropyl methylcellulose (HPMC), methacrylates, for film/enteric), Fillers/Pigments (for opacity/color, such as TiO2, talc), sugar, plasticizers (for increasing flexibility, such as polyethylene glycol).

In another embodiment, the applying the edible color additive includes dusting, spray-coating, electrostatic coating, dipping, dredging, tossing, or any combination thereof. In some embodiments, the spray-coating is an electrostatic spray-coating. In some other embodiments, the dredging or tossing can include rotating a drum tumbler with the substrate and edible color additive.

In another embodiment, the xanthommatin is separately, or in the dry-based carrier is applied onto the edible substrate by electrostatic coating.

Embodiments described herein directed to the substrate having an edible color additive within the substrate or coated on the substrate and the method of coating an edible substrate are interchangeable and not limited to each individual embodiment.

### Examples

### Pigment Aerosols and Extinction

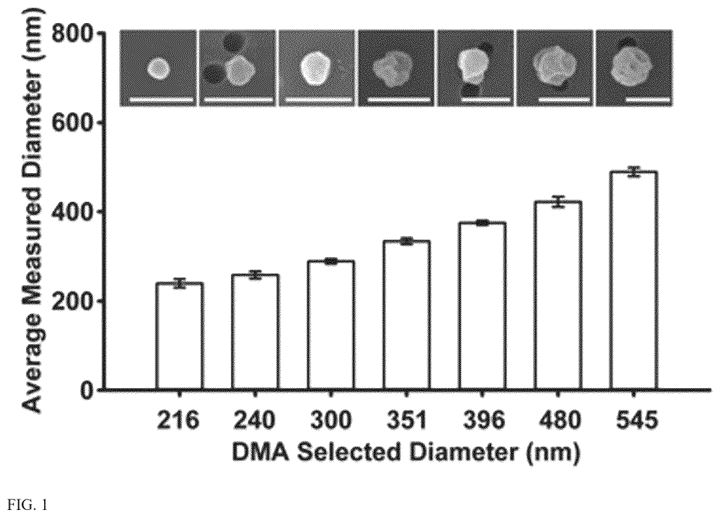

Nanostructured pigment aerosols were manufactured by nebulizing a pigment solution extracted from native squid chromatophores. It was observed that aggregates of the squid pigments (diameter range 200-600 nm) could be successfully aerosolized and collected as air and vacuum stabilized particles (FIG. 1). To accomplish this, the solution of pigments in water or methanol was nebulized (using a standard atomizer and/or spray coater) under air or inert gas such as N₂. It was found that particle diameters can be separated by their electronic mobility, suggesting a control over selected size parameters that makes this process highly tunable. FIG. 1 provides a graph that shows the average measured diameters of the pigment aerosol particles, determined from SEM images, plotted versus the DMA selected diameters. It was further observed that the concentration-dependent extinctions increase linearly with particle number concentration, indicating that the generated pigment aerosols are indeed responsible for extinguishing light.

Overall these properties, along with the pigment refractive index at a specific wavelength or range of wavelengths, are important considerations in manufacturing pigment particles for future spray-coating or air-brushing applications.

### Method for Stabilizing Pigments and Pigment Aggregates

Solutions of ommochrome pigments, pigments encapsulated within synthetic nanoparticles (e.g., silicon dioxide, titanium dioxide, and/or polymers including polyurethanes, polyesters, polyethylene glycol, or polymethacrylates), and/or native chromatophore pigment granules can be stabilized in a polyelectrolyte solution to produce, for example, uniformly casted thin films containing distributed particles and pigments.

A suspension (0.16-2.45 mg/ml) of pigmented particles and free pigment can be casted within a poly-acrylic acid (PAA) matrix onto glass slides. The positively charged PAA layer (˜60 nm thick) was used to minimize crack formation in the films through electrostatic interactions with the granules (zeta potential=−10.6 mV) upon deposition. It is believed that the PAA layer also aided in stabilizing the pigment granules, producing a uniform film. In this configuration, the hybrid films (e.g. mimetic of the natural chromatophores) included granules embedded in the polymer layer producing a ˜mm scale pigmented surface with varying hues. Pigment granule films stabilized in positively charged polymeric matrices have been formed.

### Thin Films Scattering Measurements

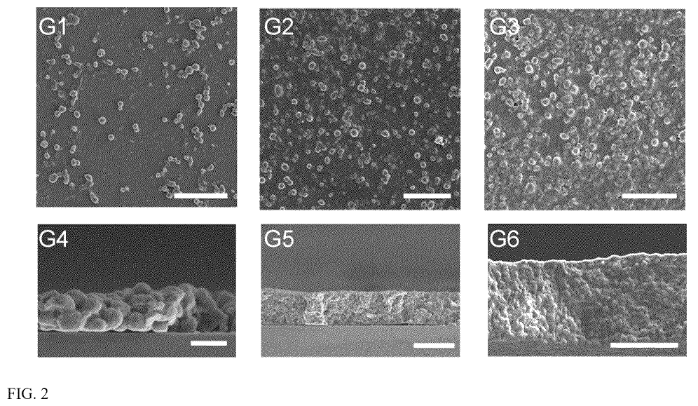

Thin films containing a heterogenous distribution of chromatophore pigment granules—either synthetic or isolated from squid (thicknesses ranging from 200 nm-7300 nm, see FIG. 2)—were formed. Briefly, 18 cm×18 cm glass slides were sonicated for 30 mins into isopropanol, ethanol and acetone prior to use. Once dried, a 8.3 wt/v % solution of polyacrylic acid (PAA, M.W. 240,000, 25 wt % solution in water; pH 3.5) was dispensed on the glass surface and spun for 1 min at 2,000 rpm to obtain a uniform coating using a G3P-8 Spin Coater. Next, a granule-water suspension (concentration was varied from 0.16 mg to 2.45 mg per ml water) was drop-casted directly onto the PAA coated glass surface and dried at 70° C. for 1 hour, until the films were dried. A Perkin Elmer Lambda 900 with the integrating sphere (which enables front and back scattering, as well as specular transmission and reflection) was used to measure the transmittance as a function of wavelength for a monolayer of granules with an inter-granular distance smaller than 3 micrometer (see FIG. 2) and a film comprising a multi-layer of pigment granules embedded within the PAA matrix. Prior to analysis, the instrument was auto-zeroed and the specular transmission of air was measured. For each film, the total integrated transmission (Tᵢₙₜ) and forward scattering (S_{f}) were measured, and the specular transmission was calculated by subtracted S_{f} from Tᵢₙₜ. The total integrated reflectance (Rᵢₙₜ) and backward scattering (S_{b}) were measured similarly used to calculate the specular reflectance. S_{b} was measured by removing a specular light port from the integrating sphere. These tunable profiles are important for enhancing the reflected or transmitted color perceived from the biomimetic/bio-compatible coatings for when administered for topical applications. Scattering is important in systems of color and must be included in future discussion of color-changing materials, or else absorption will be incorrectly determined. As in the case of clouds in front of a sunset, scattering may enhance or distort color, producing a “glowing” background with a particular color. While its exact role in the cephalopod skin is still unknown, the pigment granules contribute to nano-enabled scattering throughout the visible, near-IR, and short-wave IR regions with a brightness that is dependent on the thickness of the particle layer and/or by combining a back-reflector with a specific band pass. These characteristics make such bio-derived materials interesting candidates for future photonic devices or materials designed to color-match, camouflage, or communicate within the environment.

### Preparation of Biomimetic Nanoparticles

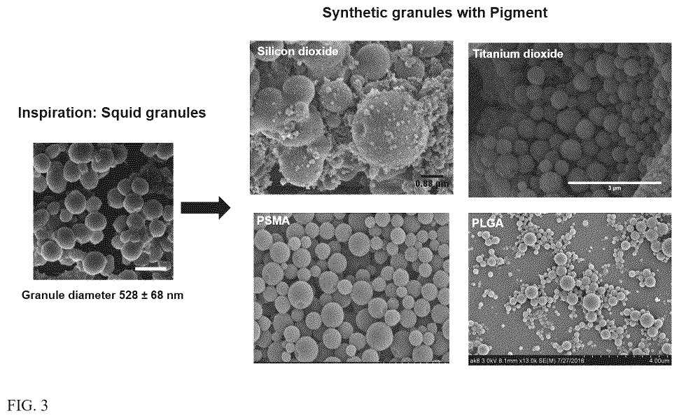

Synthetic and natural phenoxazone-based pigments have been incorporated with metal oxide and polymeric nanoparticles. Briefly, it has been demonstrated that both titanium (IV)bis(ammonium lactate) dihydroxide (TiBALDH) and tetramethylorthosilicate (TMOS) can be used a precursor solution to form TiO₂ and SiO₂ nanoparticles that stabilize the pigment structure/optical function (FIG. 3). When the precursor TiBALDH or TMOS interacts with a positively charged, amine-terminated (poly(amidoamine), PAMAM) dendrimer, an electrostatic binding event occurs in tandem with coulombic repulsion to precipitate nanoparticles. Thus, a similar electrostatic approach was used to encapsulate pigments and assay their optical performance. Synthetic particles were formed using extracted squid pigment (xanthommatin, negatively charged based on pKa values, pH ˜11) coordinated with the positively charged PAMAM dendrimer. Next, the TiBALDH was added to the mixture, causing an immediate precipitate (titanium dioxide nanoparticles) to form. This allowed to build synthetic materials that recapitulate the nanospherical structure, absorbance, and scattering of native chromatophore pigment granules for improved color properties, brightness, and hue. The use of polymers as stabilizers for the pigment aggregates was also investigated. Poly-styrene maleic anhydride (PSMA) and poly(lactic-co-glycolic acid) (PLGA) were used as polymeric stabilizers. Structurally, PSMA and PLGA feature both hydrophobic and hydrophilic regions which can lead to diverse structures formed given their ability to act as colloidal-dispersing agents using nanoprecipitation method. (FIG. 3) Dropwise addition of an organic solution containing polymer and pigment into aqueous phase resulted an instantaneous formation of nano and micro structured particles. The size could be controlled by changing the concentration of the polymer solution and the ratio between the organic and aqueous (e.g. water) phase. Size, surface characterization and photophysical properties were performed after repeated centrifugation and resuspension of particles in water. (FIG. 3) This has led to their use in pharmaceuticals to stabilize water insoluble organic molecules. In addition, the polymers feature carboxylic acid functional groups which allow for bonding of additional molecules such as drugs or pigment. In addition to structural advantages, polymer nanoparticles or PDots have been shown to not be cytotoxic in multiple cellular assays. By taking advantage of the functional groups and structural properties of PSMA and PLGA nanoparticles, their utility as stabilizing agents for the pigments was demonstrated.

### Suspension of Pigments Solutions

Pigments described herein, for example, pigments isolated from squid dermal tissue have been suspended in solutions (dimethylformamide (DMF); dimethyl sulfoxide (DMSO); formic acid; methanol; propylene carbonate (PC); methanol; and water) at concentrations ranging from 0.15-2.0 mg/mL. The absorbance of the isolated pigments suspended in various solvents has been measured using a UV-vis spectrophotometer. It has been found that the pigments are capable of absorbing a broad spectrum of UV-visible light that can be adjusted depending on the solvent used. Accordingly, the pigment alone can be used as a UV-filtering agent in methanol and buffered aqueous solutions. These properties can be enhanced and/or stabilized when the pigment is incorporated within a composition as described herein.

### Bulk Isolation of Pigments

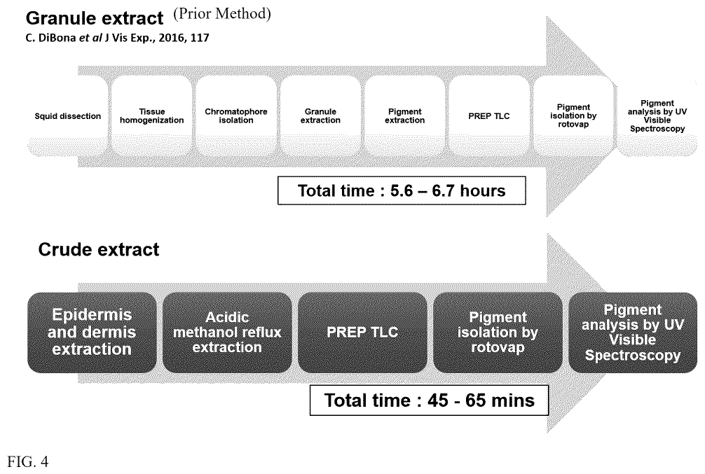

Decapitated adult squid Doryteuthis pealei are used and chromatophore pigments and pigment granules are isolated using a method based on a reflux-assisted reaction. The resultant colorant solution was collected and purified using centrifugation and size exclusion chromatography. The experiment time for this process is ˜45-75 min depending on the size of tissue and can generate ˜180 mg of pigment per squid. This process reduces time compared to manually dissected and extracted squid pigments (experiment time ˜6 hr; average pigment extracted 11 mg per squid) and is a more practical and scalable approach to the mass isolation and purification of squid dermal pigments (details in FIG. 4).

Granule extraction has been compared with reflux extraction in terms of speed of extraction and the amount of extracted pigment (details in FIG. 4).

Briefly, the granule extraction protocol involved the following steps: 1) squid dissection, 2) tissue homogenization, 3) chromatophore isolation, 4) granule extraction, 5) pigment extraction, 6) chromatography, 7) rotovap and 8) UV visible spectroscopy. The reflux assisted pigment extraction protocol involved the following steps: 1) epidermis extraction, 2) acidic methanol reflux extraction at 66° C. for 30 min, 3) size exclusion chromatography, 4) rotary evaporation to remove an excess solvent, and 5) UV visible spectroscopy and mass spectrometry to verify pigment composition.

Using the above described granule extraction method, an average amount of pigment per squid of 11 mg was extracted within 5.6 to 6.7 hours. Using the above described reflux assisted extraction method, an average amount of pigment per squid of 183 mg was extracted within 45 to 75 minutes. Thus, it was found that reflux extraction was 6.5 times faster and produced 16 times more pigment than granule extraction.

### Electrosynthesis of Ommochromes

The oxidative cyclization of 3-hydroxykynurenine has previously been reported using a procedure adapted from Budendat and coworkers (see Butenandt, A.; Schäfer, W., Recent Progress in the Chemistry of Natural and Synthetic Coloring Matters and Related Fields. Academic Press: New York, 1962). Briefly, 2 units of 3-hydroxykynurenine are suspended in 0.1M K-phosphate buffer (2 mL, pH=7.0). The solution is reacted with 0.1M potassium ferricyanide at room temperature for 10 min, whereupon an orange pigment mixture containing xanthommatin is produced. While referenced in the literature, this method routinely produces a low (˜20% yield) with low purity of material.

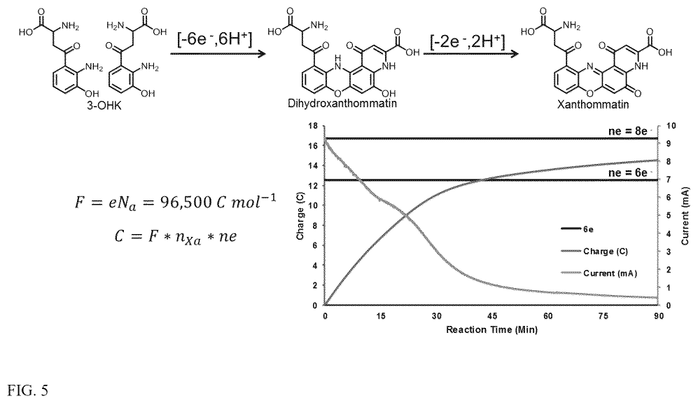

It has now been found that xanthommatin can be produced in bulk using electrochemical oxidation. The precursor compound 3-hydroxykynurenine, purchased from Sigma Aldrich, is incorporated in an electrolyte bath. Instead of an oxidizing agent, it is possible to apply an oxidizing current using a potentiostat. Since the color changes upon oxidation, one can follow the reaction spectroscopically (FIG. 5).

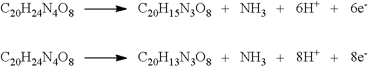

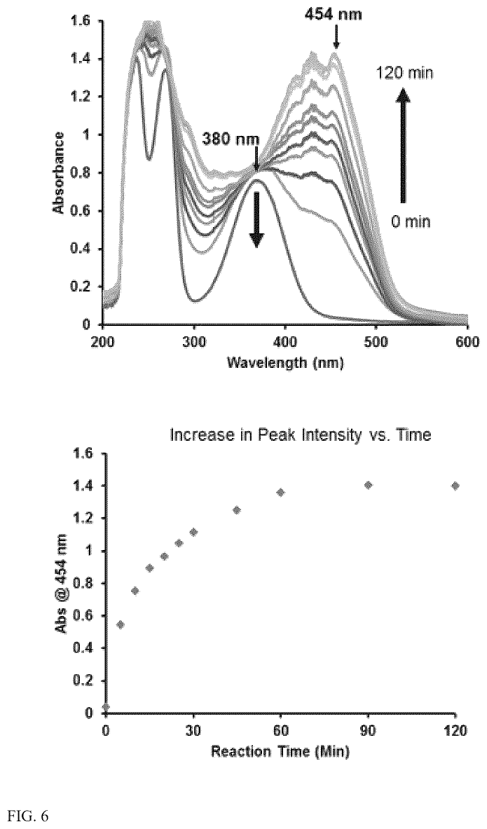

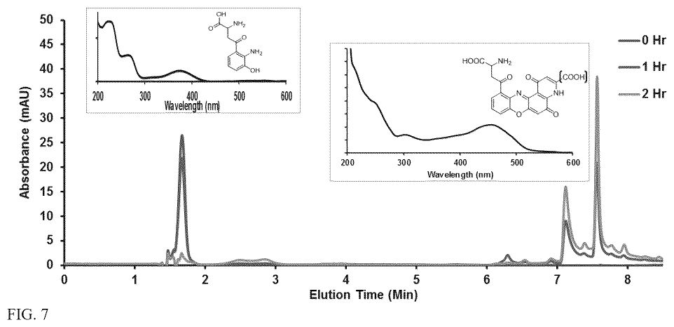

Setup: Electrochemistry was performed with Reticulated Vitreous Carbon (Glassy Carbon Foam), a high surface area working electrode, Pt counter, and Ag/AgCl reference electrodes in beaker. An oxidizing potential of 0.6-1.2 V was applied, and the current integrated over ˜100 minutes. This was a measure of how much charge passed through the working electrode. For an ideal/complete redox reaction: Q=(moles of analyte)*(# of electrons)*(Faraday constant). The expected Q values are shown along with the measured Q for the reaction (with 6 or 8 electrons consumed depending on whether xanthommatin is oxidized or reduced). Assuming the product is (oxidized xanthommatin), the chronocoulometry (FIG. 5), UV-vis spectrophotometry (FIG. 6), and high-performance liquid chromatography (FIG. 7) shows that *up to*97% of the 3-OHK is oxidized, which is the highest reported yield for this reaction to date.

### pH and Voltage-Sensitivity of Xanthommatin

It has been found that xanthommatin can be used as a pH- and voltage-sensitive biochrome. At a neutral pH, solutions of xanthommatin exhibit a deep-orange color, and under acidic (pH<3) conditions, the color was found to diminish. Based on Density Functional Theory (DFT) calculations and further supported with electrochemical measurements, it is believed that xanthommatin changes both structurally and functionally dependent on its protonation state.

The purified xanthommatin (˜5 mg) was suspended in water (5.00 mL) and titrated to a starting pH of 2.08. The solution was then titrated with 0.05 M NaOH until the pH reached 10.37. During each point in the titration, the solution pH and absorbance was determined. pH levels were measured using a Fisher Scientific Accumet AP110 PH meter (Fisher Scientific, Waltham, MA).

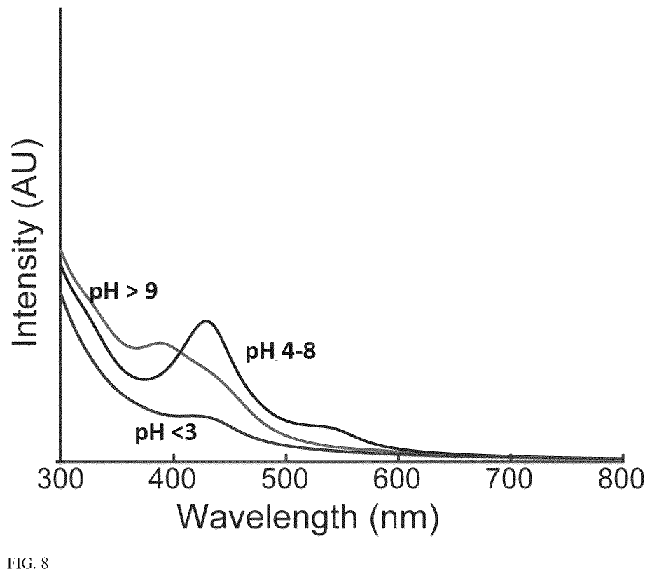

The pH-dependent color change of a 2.4×10⁻⁶ mM solution of xanthommatin was monitored over a pH range of 1.90-10.30 using spectrophotometry. Three distinct patterns were associated with the increasing pH (see FIG. 8). At pH<3.00, the pigment was pale-yellow with a λₘₐₓ˜430 nm. Then at pH ˜3.60, the solution transitioned to a darker color which ultimately saturated at pH 7.70. This intensity was maintained until pH ˜8.30, whereupon the peak at 430 nm decreased, and the presence of a secondary peak at 360 nm emerged. MSMS analysis was used to identify that pigment hydrolysis occurred when pH values approached and exceeded 10.00 via a mass addition of water that persisted throughout ionization. Thus, for the remaining experiments, a pH of 8.30 was not exceeded. To determine whether the observed intensity in color was reversible at the lower pH values, the pH of xanthommatin was next cycled between acidic (2.00-3.00) and neutral (pH 6.00-8.00) conditions six times, and the absorbance intensity at 430 nm was followed. A good switching behavior was observed from the pale yellow to the dark orange over 12 total acquisitions. Collectively, these data suggest a dependency of the pigment's color intensity to its protonation state.

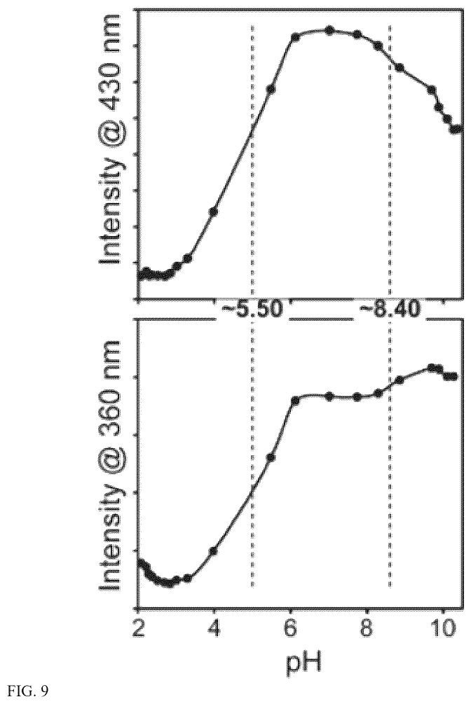

If the presence or absence of protons were indeed influencing the resultant visible color, then individual acid dissociation constants (Kₐ) could be experimentally extrapolated and used to follow compositional changes in the biochrome as a function of pH. To test this, it was determined how absorbance intensities varied as a function of pH at both 430 and 360 nm (see FIG. 9). Using the combination and normalization of the Henderson-Hasselbalch and Beer-Lambert laws (see Braun, R. D., Introduction to Chemical Analysis. 1982, 197-199; and Son, Y. A.; Kim, S.-H., New pH indicator based on 1,3-bisdicyanovinylindane. Dyes Pigm. 2005, 64, 153-155), the sharp increases in intensity at the two wavelengths were extrapolated as unique pKₐ values. These points were illustrated at the center of the least pH change as the point where pKₐ=pH. Extrapolated yielded pKₐ values of 5.50 and 8.40 (dashed lines in FIG. 6C). Using advanced chemistry development (ACD) predictions, it was determined that the neutral condition (5.50<pKa <8.40) elicited protonation of the phenoxazine amine. A pH<3.00 was associated with protonation of the carboxylic acids yielding an overall+3 charge to xanthommatin, and the pKa >8.40 was associated with the deprotonation of amino acid amine and oxazine amine yielding an overall −2 charge. Together, these data indicated that the pigment charge influences its absorptive properties.

### The Redox-Dependent Color Change

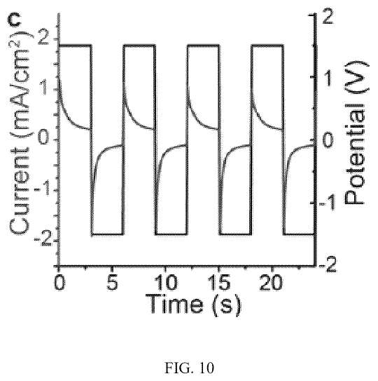

Even though its exact mechanism for electron transfer remains unknown, phenoxazone-based materials are believed to undergo a reversible reduction to dihydroxanthommatin, gaining two electrons and two protons during its redox-dependent color change. These principles are used to show how the pigment stabilized in a polymer (PEDOT-PSS) matrix is capable of switching color in response to different applied voltages. When a double potential step from +1.5 V to −1.5 V vs ground was applied to the device, a redox-dependent color switch was observed (FIG. 11). The current required to switch from one colored state to another was plotted as a function of time upon application of a square-wave potential (FIG. 10). The device with a ˜4 cm² active area exhibited a response time approaching ˜2 sec to switch from the reduced to oxidized form, and ˜1 sec to achieve the reverse reaction (FIG. 10). In each case, the switching time for each step was calculated as the time required to reach 95% of the maximum current. These features were comparable to the current state of the art of electrochromic devices (ECDs) which also exhibited switching speeds on the order of ˜sec.

The spectral tunability of these materials was also investigated by varying the ratio between PEDOT:PSS and pigment (FIG. 11). Different concentrations of xanthommatin (from 0.04 mg/ml to 0.16 mg/ml) were used while the amount of PEDOT:PSS remained constant. The color of each film activated at the different redox states was measured using the International Commission of Illumination (CIE) 1931 xy-chromaticity diagram. The first system (O1 and R1), which contained 0.01 mg Xa/cm², generated a neutral color in both oxidized (x=0.3197, y=0.4080) and reduced (x=0.3095, y=0.3197) states. On the other hand, by increasing the amount of Xa (0.4 mg Xa/cm²) in the films, richer colors were achieved in both oxidized (O3; x=0.4555, y=0.4371) and reduced (R3, x=0.4300, y=0.3600) conditions. The spectra of each redox state were also collected and plotted in FIG. 11, where the optical contrast (ΔT %=% T_{oxidized}−% T_{reduced}) between the two-color states (reported in Table 1) was extrapolated.

TABLE 1
The CIE 1976 color space for the pigment granule films, including
calculated C*_{ab} and saturation values, as a function of thickness,
determined by SEM cross-sectional imaging. The standard range
for L* and saturation span 0-100%.

| | | | | | | Thickness | Granule |
|---|---|---|---|---|---|---|---|
| Sample | L* | a* | b* | C*_{ab} | Saturation | (nm) | Layers |
| G1 | 75.2 | 1.7 | 2.4 | 2.9 | 3.8 | — | ~<1* |
| G2 | 45.2 | 4.1 | 4.8 | 6.3 | 13.9 | — | ~<1* |
| G3 | 43.3 | 16.3 | 9.8 | 19.1 | 40.2 | 530 ± 160 | ~1 |
| G4 | 42.9 | 14.6 | 7.4 | 18.1 | 38.8 | 1170 ± 150 | ~2 |
| G5 | 12.2 | 12.5 | 1.5 | 11.4 | 68.3 | 3880 ± 150 | ~7 |
| G6 | 7.6 | 8.5 | −0.1 | 6.2 | 63.2 | 5820 ± 430 | ~12 |
| WS-1 | 99.7 | 0.0 | 0.0 | 0.0 | 0.0 | — | — |

*at less than 1 monolayer, thickness of the granule film is not well-defined.

Because the human eye is most sensitive at 555 nm, all transmittance values were calculated at this wavelength without any background correction. For darkest yellow (03) to red (R3) films, a 25.4% (69.5% T to 44.1% T) optical contrast was observed, indicating that indeed ECDs assembled using Xa can reversibly switch into two distinct colors at low operational voltages.

This process can also be replicated in the absence of an applied electrochemical field. Instead of using voltage and PEDOT-PSS as a stabilizer, pigments can be coated directly on soft tissue or synthetic tissue (e.g. hydrogels) in one color (yellow state) which can easily and quickly (within ˜1 min) switch to a red color that is stable over 120 hrs in air upon application of a mild reducing agent (e.g. ascorbic acid). This demonstrates the color-switching capability of the phenoxazone/phenoxazine-based pigments for color changing topical treatments.

### UV-Filtering Properties of Xanthommatin-Based Materials

Natural dyes and pigments often have functions that extend beyond simple pigmentation. For instance, the chlorophyll in chloroplasts absorbs light at 430 and 660 nm producing a green color in plants but is also used in conjunction with water and carbon dioxide to produce adenosine triphosphate, ultimately converting absorbed light into energy. On the other hand, eumelanin, found in most living organisms, is black in color with a broadband absorption spanning the ultraviolet through visible (UV-vis) spectrum that also converts photon energy into harmless heat. Ommochromes, such as xanthommatin (Xa) and its decarboxylated form (DCXa), are the predominant pigments present in the skin of the squid Doryteuthis pealeii and are also found in the skin and eyes of arthropods and other invertebrates. As metabolites of tryptophan, these phenoxazone-based biochromes are redox active and absorb UV-vis light to display yellow or red colors that can be tuned dependent on their local microenvironments. For example, ratios of the reduced (red) and oxidized (yellow) forms of Xa and DCXa change throughout the life-cycle of the red dragonfly, indicating a sex-specific maturation in these insects. On the other hand, the squid D. pealeii contains different ratios of Xa and DCXa that combine and contribute to a range of colors used during camouflage.

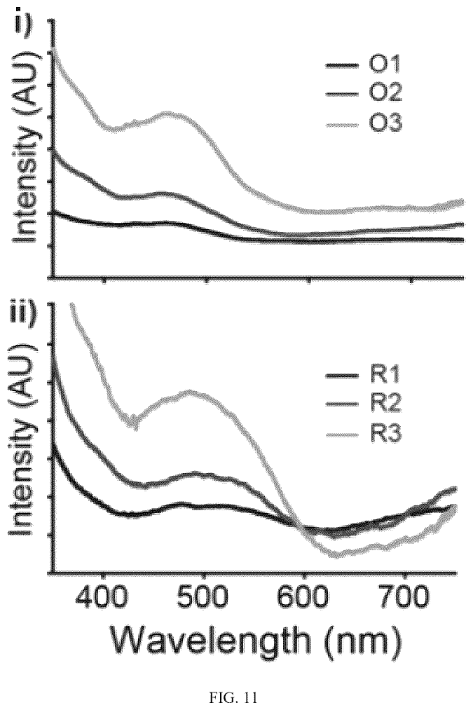

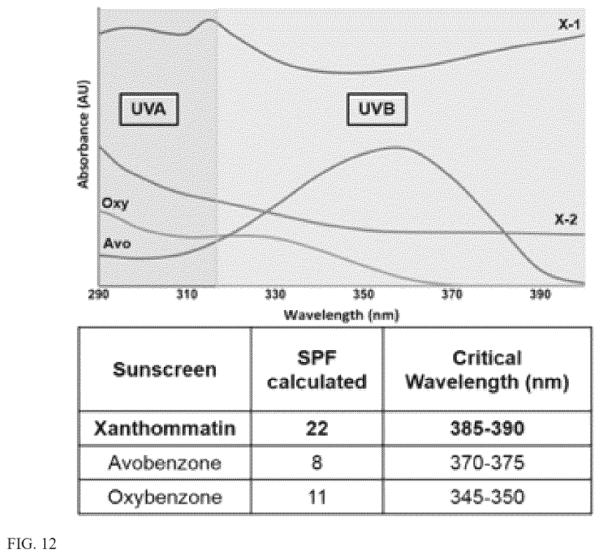

Xa is the simplest and most common ommochrome and is formed (along with the reduced dihydro-xanthommatin and ommatin subclasses) via the condensation of two hydroxykynurenine residues in the kynurenine pathway of tryptophan metabolism. Once synthesized, Xa is stabilized within granules (diameter 530 nm) that are resistant to photodegradation. An added predicted functionality, although largely unexplored in vitro and in vivo, is that Xa behaves as a free-radical scavenger. Together, with the data presented in FIG. 12 showing that Xa absorbs UV radiation ˜2× higher than current chemical filters at the same concentrations, it is expected that Xa and Xa-based materials can function as UV-filters that can not only protect against solar irradiation but can also be used in preventative skin care as free-radical scavengers (while maintaining biocompatibility).

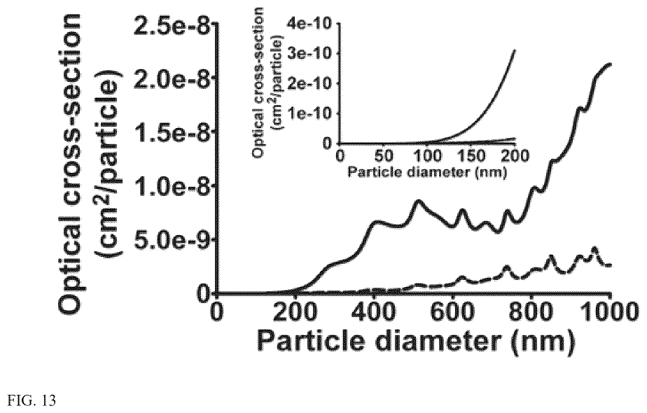

Given its propensity to form granules in natural systems (FIG. 3, left) and its impressive UV-absorptive properties in vitro (FIG. 12, see Table 2 below), Xa was encapsulated within biodegradable particles designed to improve its durability and use as a topical UV-filter. The complex refractive index (RI) of Xa was experimentally determined as 1.92+0.014i. This uniquely high RI is important, especially when considering the design criteria of the particle based UV-filters. For instance, when assembled into nano- and micro-particles, Xa will scatter more light than it absorbs at diameters >200 nm, and this difference is maximized at sizes >1 μm (FIG. 13). Because light scattering is an important feature for heat dissipation and reflection for coatings, it can be preferable to design particles with diameters ranging from 1-10 μm.

TABLE 2
Concentration dependent SPF values
calculated from Xa in vitro, representing
the upper and lower concentrations used.

| SPF | Concentration | SPF |
|---|---|---|
| Range | (mM) | in vitro |
| 2-12 | 0.062 | 3 |
| 12-30 | 0.125 | 14 |
| 30> | 0.250 | 38 |

### ROS-Scavenging Properties of Xanthommatin-Based Materials

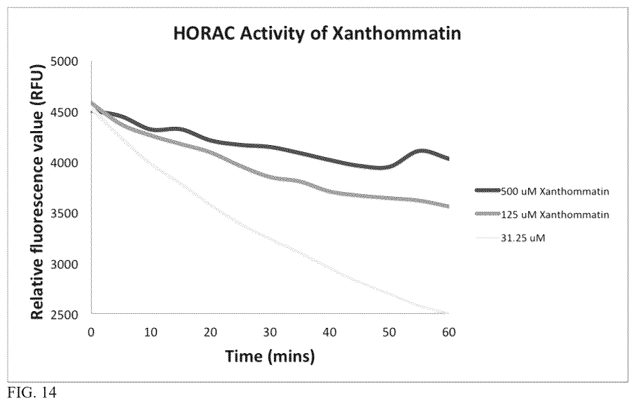

Oxygen derived species such as superoxide radical, hydrogen peroxide, singlet oxygen and hydroxyl radical are well known to be cytotoxic and have been implicated in the onset of a wide array of human diseases. These “compounds” are often referred to as reactive oxygen species (ROS). ROS normally exist in all aerobic cells in balance with biochemical antioxidants; however, this critical equilibrium can be disrupted due to excess ROS or antioxidant depletion and can result in oxidative stress. Antioxidants are used in cosmetics, pharmaceuticals, food and on the skin directly minimize the undesirable reactions resulting from oxidation caused by ROS. Commonly used antioxidants typically contain a combination of phenol, benzene and alkene functional groups. These key features work to inhibit the propagation of ROS through resonance stabilization of free radicals. Phenoxazone, phenoxazine, and a derivative or precursor thereof are great candidates for natural antioxidants due their characteristic benzene and phenol groups. The ability of particles and pigments (free and encapsulated) to generate ROS are measured using the colorimetric hydroxyl radical antioxidant capacity (HORAC) assay (FIG. 14). These xanthommatin-based materials consume the hydroxyl radicals produced by the activation of the fenton reagent preventing the degradation of the fluorescein probe. Xanthommatin showed enhanced activity when compared to commercially available antioxidant gallic acid.

### Xanthommatin as a Color Additive

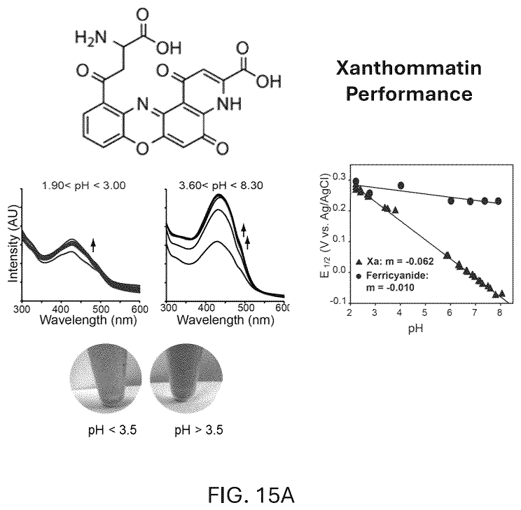

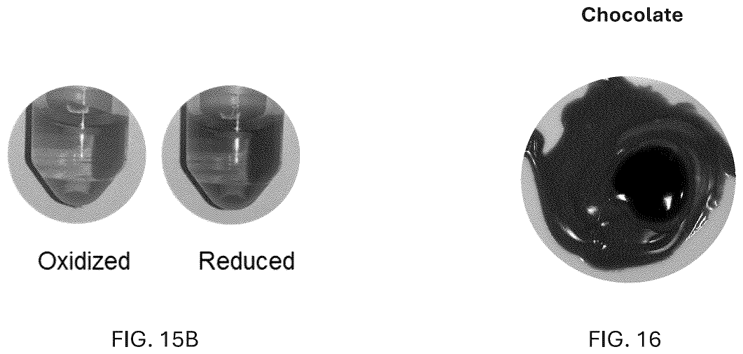

Xanthommatin was dissolved in solution at a concentration of 1 mg/mL in water. The pH was adjusted from 1.90 to 8.30 and the solution was observed to be yellow at less than pH 3.5 and red at greater than pH 3.5 (FIG. 15A). Xanthommatin was further oxidized and reduced in solution and was found to be yellow when oxidized and red when reduced (FIG. 15B).

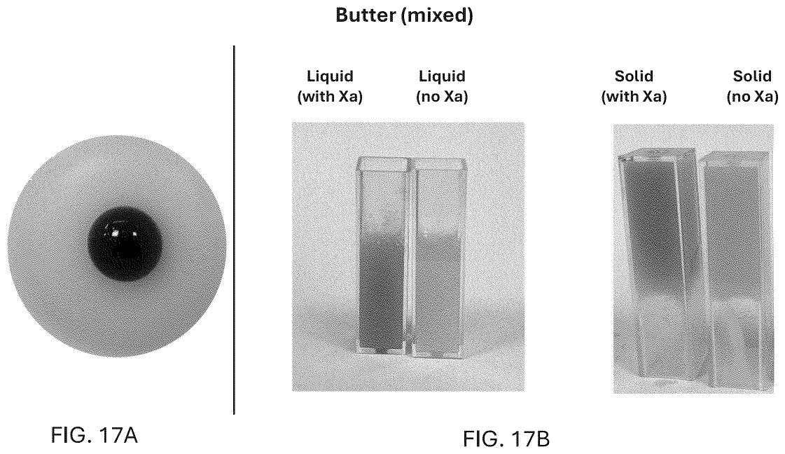

The xanthommatin solution (1 mg/mL) was then mixed into a food matrix, of either melted chocolate (FIG. 16) or melted butter (FIG. 17A). The addition of xanthommatin to butter in both melted and solidified butter (FIG. 17B) resulted in a darkened color of the butter to a brown.

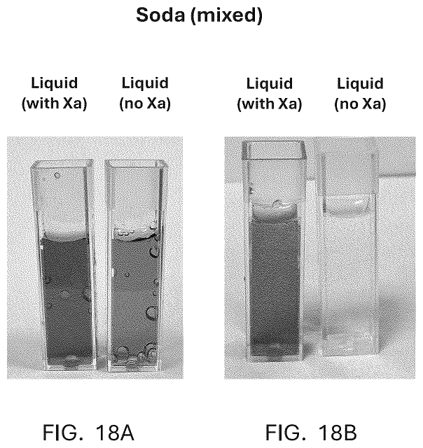

Xanthommatin solution (at a concentration of 1 mg/mL) was added to a liquid food matrix of soda (FIGS. 18A and 18B). The addition of xanthommatin to a brown soda resulted in a darkening (brown to amber) of the soda as shown in FIG. 18A. Similarly, the addition of xanthommatin to a clear soda resulted in darkening (colorless to brown) of the soda as shown in FIG. 18B. The pH of the sodas were measured at a pH of 2.4 to pH 2.5. The darkening could be adjusted by decreasing the concentration of xanthommatin added.

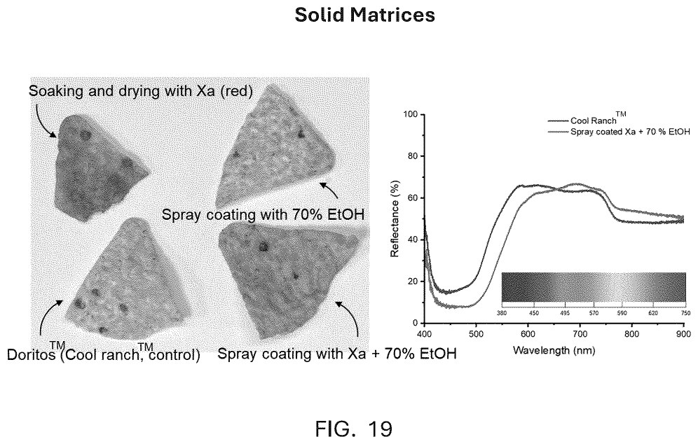

Application of xanthommatin as a coating was also explored. A solution of xanthommatin in water (1 mg/mL) was applied to a DORITOS™ Cool Ranch™ chips by soaking the chip in the xanthommatin solution and drying. Additionally, a 70% ethanol to 30% water solution was made and spray-coated onto a chip as a control. Another solution of 70% ethanol to 30% water solution was prepared with 1 mg/mL xanthommatin and also spray-coated onto a chip. As shown in FIG. 19, the soaked and dried chip appeared more red than the control chip, and the spray-coated chip with xanthommatin was slightly more red appearing than the two control chips. The reflectance spectrum further confirms this observation, indicating a red shift in the spray-coated chip.

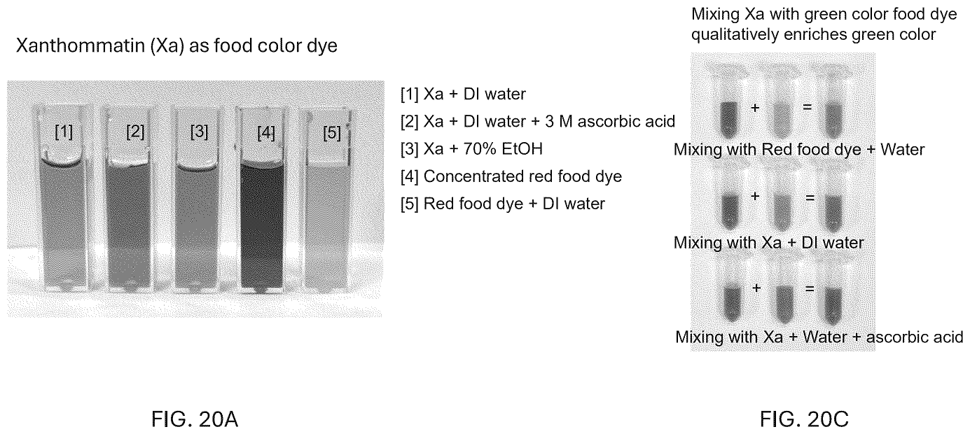

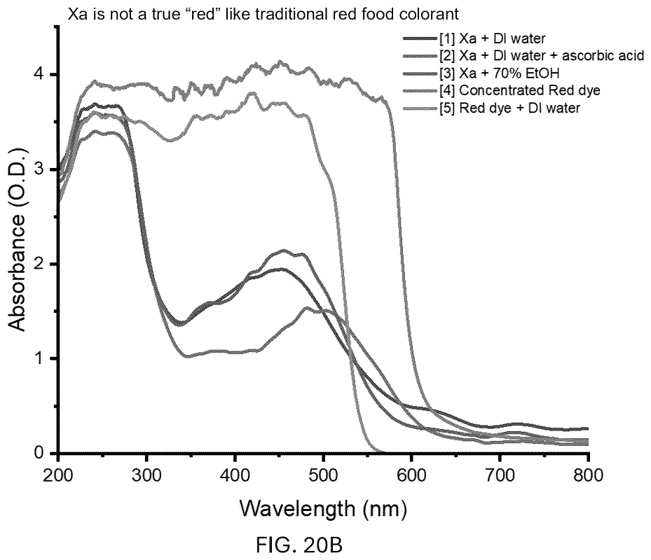

Xanthommatin (at a concentration of 1 mg/mL) was further explored as a food color dye by comparing xanthommatin (Xa) in deionized (DI) water, Xa in DI water with 3 M ascorbic acid, Xa with 70% ethanol to 30% water, and compared to concentrated red food dye and diluted red food dye (FIG. 20A). There were observable differences between the Xa solutions and red food dye, although this can be modified based on the concentration of Xa and pH of the solution that Xa is present in. The absorbance spectra (FIG. 20B) further indicates that Xa is not a true red like a traditional red food colorant. Additionally, the Xa solutions were added to a green food dye which enriched the green color of the green food dye comparatively to the mixing of the red food dye with the green food dye (FIG. 20C).

### Coating for Flavored Corn Chips

For making a corn chip, corn masa (corn dough) is formed into triangles, fired until crispy, and allowed to cool slightly. The chips emerge from a fryer hot and oil-coated, which aids in the coating to adhere to the chips.

The warm chips move through a rotating drum tumbler when the seasoning blend is applied. The process involves an electrostatically coating the mixture of the seasoning blend and edible color coating powder to stick to the chips uniformly. Other means include oil misting by spraying a light layer of oil to enhance adhesion to the chips and/or controlled dispensing by adding a specified amount of seasoning that is released into the tumbler for consistent coverage.

The seasoning blend is a powder that is engineered for optimal adhesion that has flavor compounds (e.g., cheese powder, spices, monosodium glutamate (MSG)) maltodextrin, which aids in the powder to stick to the chips and carries flavor, salt, sugar, anti-caking agents for powder flow, and edible color additives for visual appeal.

The teachings of U.S. Published Application No. 2015/0329604, titled: PIGMENT STRUCTURES, PIGMENT GRANULES, PIGMENT PROTEINS, AND USES THEREOF; and Int'l Application No. PCT/US2013/072311, titled: PIGMENT STRUCTURES, PIGMENT GRANULES, PIGMENT PROTEINS, AND USES THEREOF, now published as Int'l Publication No. WO 2014/085641, are incorporated by reference in their entirety.

The teachings of all patents, published applications and references cited herein, are incorporated by reference in their entirety.

While example embodiments have been particularly shown and described, it will be understood by those skilled in the art that various changes in form and details may be made therein without departing from the scope of the embodiments.

What is claimed is:

1. A substrate having an edible color additive within the substrate or coated on the substrate, comprising xanthommatin as the edible color additive present at a concentration of about 0.01% wt to about 0.5% wt, and optionally a dry-based carrier or a solution-based carrier.

2. The substrate having an edible color additive within the substrate or coated on the substrate of claim 1, wherein the substrate is a food, beverage, drink, medication or a drug form.

3. The substrate having an edible color additive within the substrate or coated on the substrate of claim 1, wherein the dry based carrier comprises cheese powder, spices, monosodium glutamate, maltodextrin, salt, sugar, anti-caking agents, other edible color additives, or a combination thereof.

4. The substrate having an edible color additive within the substrate or coated on the substrate of claim 3, wherein anti-caking agents include silicon dioxide (SiO₂), calcium silicate (CaSiO₃), magnesium carbonate (MgCO₃), magnesium silicate (MgSiO₃), sodium aluminosilicate (Na₂Al₂Si₁₄O₃₂·3H₂O), talc (Mg₃Si₄O₁₀(OH)₂), tricalcium phosphate (TCP, Ca₃(PO₄)₂), or a combination thereof.

5. The substrate having an edible color additive within the substrate or coated on the substrate of claim 1, wherein the solution-based carrier comprises water, glycerin, propylene glycol, oils, alcohols, or a combination thereof.

6. The substrate having an edible color additive within the substrate or coated on the substrate of claim 5, wherein the solution-based carrier is about 50% v/v ethanol to about 50% v/v water to about 80% v/v ethanol to about 20% v/v water.

7. The substrate having an edible color additive within the substrate or coated on the substrate of claim 1, wherein the edible color additive absorbs light in a wavelength range of about 420 nm to about 480 nm.

8. The substrate having an edible color additive within the substrate or coated on the substrate of claim 1, wherein the edible color additive absorbs light in a wavelength range of about 620 nm to about 750 nm.

9. The substrate having an edible color additive within the substrate or coated on the substrate of claim 1, wherein the edible color additive absorbs light in a wavelength range of about 590 nm to about 620 nm.

10. The substrate having an edible color additive within the substrate or coated on the substrate of claim 1, wherein the color additive is a coating on the substrate.

11. The substrate of claim 10, wherein the substrate is a food, medication, or drug form.

12. A method of coating an edible substrate, the method comprising:
applying xanthommatin as an edible color additive at a concentration of about 0.01% wt to about 0.5% wt, and optionally a dry-based carrier or a solution-based carrier.

13. The method of claim 12, wherein the applying the edible color additive comprises dusting, spray-coating, electrostatic coating, dipping, dredging, tossing, or a combination thereof.

14. The method of claim 12, wherein the coating absorbs light at a wavelength range of about 420 nm to about 480 nm.

15. The method of claim 12, wherein the coating absorbs light at a wavelength range of about 620 nm to about 750 nm.

16. The method of claim 12, wherein the coating absorbs light at a wavelength range of about 590 nm to about 620 nm.

17. The method of claim 12, wherein the xanthommatin is separately applied, or applied in combination with the dry-based carrier onto the edible substrate by electrostatic coating.
